(19) United States
(12) Patent Application Publication
(10) Pub. No.: US 2022/0133792 A1
(43) Pub. Date: May 5, 2022

(54) SIGNALING PLATFORMS FOR CHIMERIC ANTIGEN RECEPTOR T CELLS

(71) Applicant: THE TRUSTEES OF DARTMOUTH COLLEGE, Hanover, NH (US)

(72) Inventors: Charles SENTMAN, Grantham, NH (US); David GRABER, Hanover, NH (US); W. James COOK, Hanover, NH (US)

(21) Appl. No.: 17/427,117

(22) PCT Filed: Jan. 31, 2020
(86) PCT No.: PCT/US20/16127

§ 371 Date: Jul. 30, 2021

Related U.S. Application Data
(60) Provisional application No. 62/799,924, filed on Feb. 1, 2019.

(51) Int. Cl. A61K 35/17; C07K 14/725; C07K 14/705; A61P 35/00

(52) U.S. Cl. CPC A61K 35/17; A61P 35/00; C07K 14/70521; C07K 14/7051

(57) ABSTRACT

Chimeric antigen receptors (CARs) are disclosed, along with nucleic acids and vectors encoding, and recombinant cells comprising such CARs and therapeutic compositions containing any of the foregoing. The CARs may comprise mutations that alter CAR expression, cytotoxicity, or cytokine production. Also provided are methods for using recombinant cells comprising these CARs for immunotherapy, e.g., in treating cancer by the administration of a therapeutically effective amount of one or more of the CAR polypeptides, nucleic acids, vectors, and/or immune cells, e.g., human CAR T cells, described herein optionally in combination with other immune and cancer treatments.

### RELATED APPLICATIONS

This application claims benefit of priority to U.S. provisional application No. 62/799,924 filed Feb. 1, 2019, the contents of which are incorporated by reference in their entirety.

### STATEMENT REGARDING FEDERALLY SPONSORED RESEARCH OR DEVELOPMENT

This invention was made with government support under Grant No. P30GM103415-15 awarded by the National Institutes of Health. The government has certain rights in the invention.

The sequence listing in the file named “1143252o003213.txt” having a size of 3,871 bytes that was created Jan. 30, 2020 is hereby incorporated by reference in its entirety.

### FIELD OF THE INVENTION

The invention disclosed herein relates to how the specific design elements of intracellular signaling domains change the effector responses of chimeric antigen receptors (CARs) and compositions containing same, and uses of such CARs. In particular, there are provided chimeric antigen receptors comprising modified intracellular signaling domains. Nucleotide sequences encoding, and amino acid sequences comprising such CAR constructs are also disclosed. Vectors comprising the nucleic acids encoding such constructs, cells expressing such constructs, pharmaceutical compositions, and methods of making and using such compositions are also provided.

### BACKGROUND OF THE INVENTION

Chimeric antigen receptors (CARs, also known as chimeric immunoreceptors, chimeric T cell receptors or artificial T cell receptors) are receptor proteins that have been engineered to give immune cells the ability to target a specific protein. The receptors are chimeric because they combine both antigen-binding and T-cell activating functions into a single receptor. Chimeric antigen receptors (CARs) are typically composed of three basic parts: a recognition or antigen targeting domain, a transmembrane domain, and one or more signaling domains (Sadelain, et al. (2013) Cancer Discov. 3:388-398; Park, et al. (2011) Trends Biotechnol. 29:550-557). The recognition domain can be based on an antibody, a T cell receptor, another receptor, or a ligand for a receptor. The transmembrane domain includes an extracellular stalk region and may allow for dimerization. The signaling portion involves a protein domain that induces a primary activation signal in cells (e.g., CD3-ζ or FcεRIγ).

CAR T cell therapy represents an emerging type of immunotherapy whereby patient lymphocytes are genetically modified to express a receptor that allows recognition of a specific antigen. Upon antigen recognition, these modified T cells are activated via signaling domains converting them into potent cell killers. CAR-transduced T cells have been shown to constitute an effective means to eliminate tumors and increase patient survival (Sadelain, et al. (2009) Curr. Opin. Immunol. 21:215-23; Sadelain, et al. (2003) Nat. Rev. Cancer 3:35-45; Barber, et al. (2008) J. Immunol. 180:72-78).

The expansion and persistence of CAR T cells has been improved by the development of ‘second-generation’ CARs which are engineered to comprise a costimulatory endodomain derived from costimulatory molecules such as CD28 and 4-1BB. T cells expressing these CARs retain their cytotoxic function, and upon antigen engagement produce a variety of cytokines, such as IFN-gamma, TNF-alpha, GM-CSF, and interleukin-2 (IL-2), which helps to sustain their activation and expansion (Vera, et al. (2006) Blood 108:3890-7; Kowolik, et al. (2006) Cancer Res. 66:10995-11004; Maher, et al. (2002) Nat. Biotechnol. 20:70-75), as well as enhancing their antitumor activity (Sadelain, et al. (2009) Curr. Opin. Immunol. 21:215-223; Vera, et al. (2006) Blood 108:3890-7; Kowolik, et al. (2006) Cancer Res. 66:10995-11004).

### BRIEF SUMMARY OF THE INVENTION

The present invention relates to chimeric antigen receptors comprising one or more mutations that alter the functional characteristics of the CARs or the cells comprising such CARs. A particular aspect of the invention is directed to CARs comprising mutations in the co-stimulatory domains. Such mutations may affect CAR T cell cytotoxicity, expression, and/or cytokine production, among other features.

### DETAILED DESCRIPTION OF THE SEVERAL VIEWS OF THE DRAWINGS

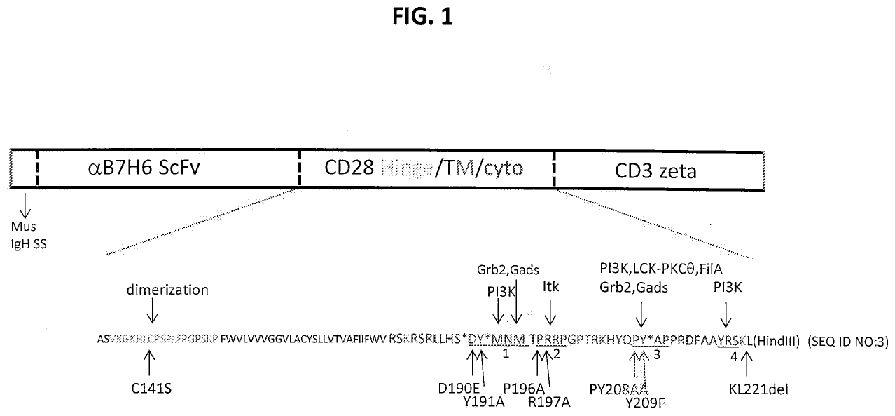

FIG. 1 contains a general schematic of an exemplary anti-B7H6 CAR comprising a CD28 co-stimulatory domain. The schematic illustrates exemplary domains, motifs, and mutations.

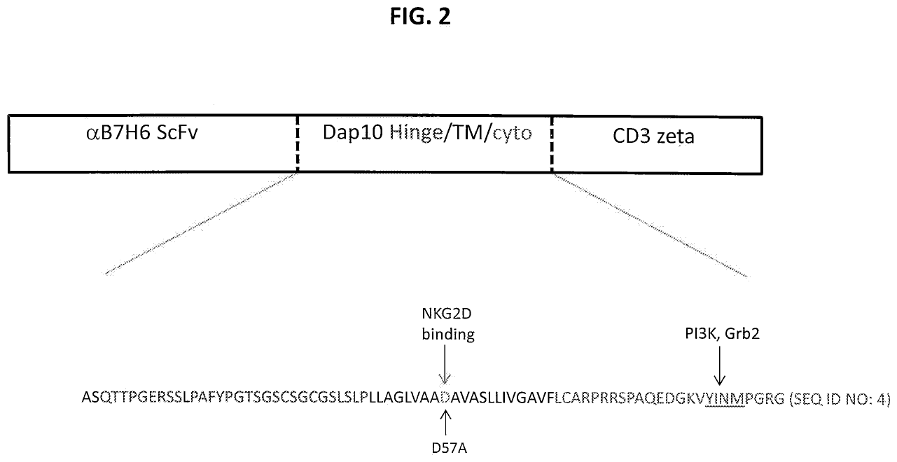

FIG. 2 contains a general schematic of an exemplary anti-B7H6 CAR with a DAP10 co-stimulatory domain. The schematic illustrates exemplary domains, motifs, and mutations.

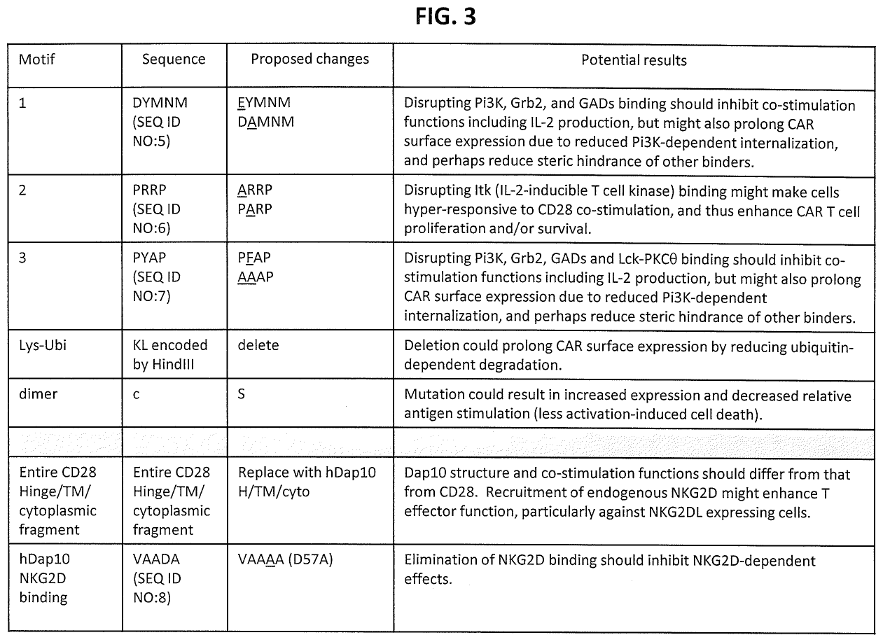

FIG. 3 contains a table naming various co-stimulatory domain sequence motifs, as designated in FIG. 1 and FIG. 2, along with their amino acid sequences and proposed modifications of those sequences, along with the potential results of those modifications.

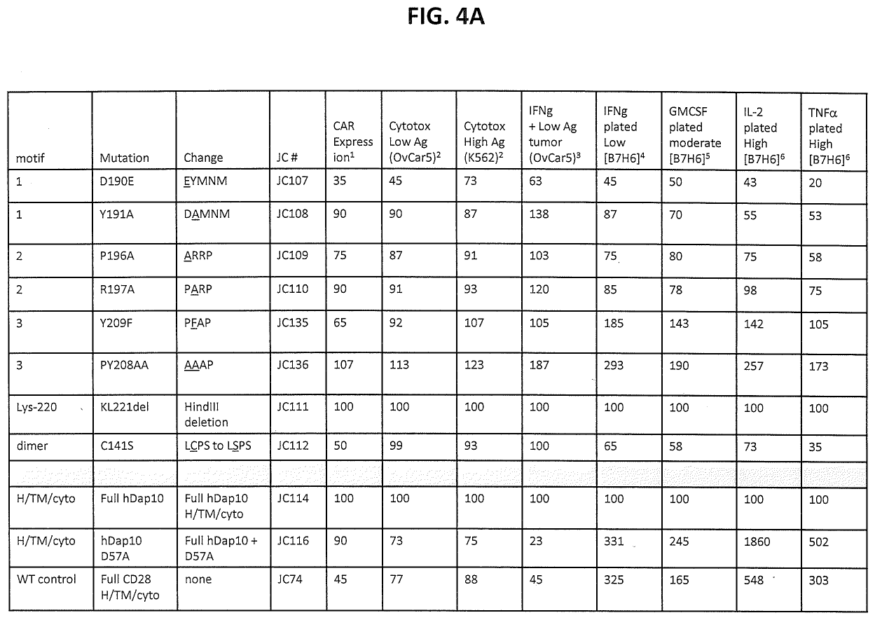

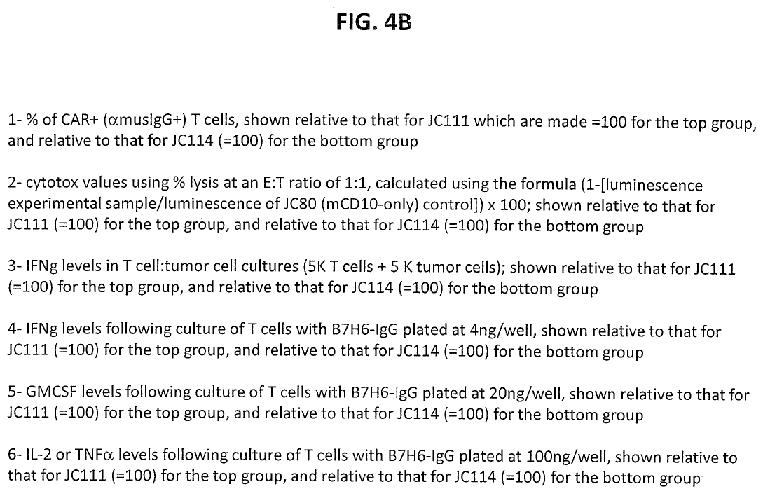

FIGS. 4A-B contain the summary of in vitro testing results of exemplary CAR constructs. FIG. 4A contains a table with the results of exemplary CAR construct testing. FIG. 4B contains a listing of the experimental conditions for the results portrayed in FIG. 4A.

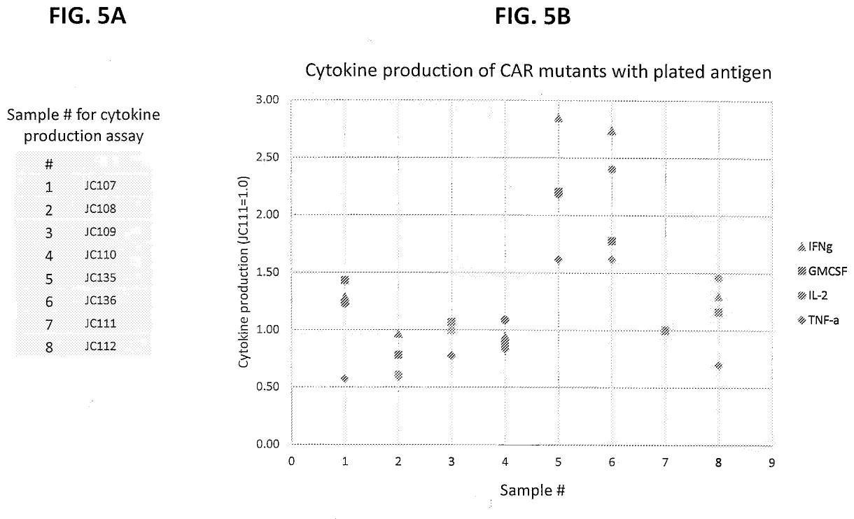

FIGS. 5A-B contain data summarizing the relative cytokine production from T cells comprising various CAR constructs. FIG. 5A provides the key indicating the sample number for each of the CAR constructs tested. FIG. 5B provides the results of the cytokine production assays, normalized for percent CAR expression in order to provide the cytokine production on a per cell basis. The results are then given relative to the results from T cells transduced with construct JC111, sample #7, which was set to 1.0. (Note that each of the four markers indicating the cytokine production for sample #7 overlap at 1.0).

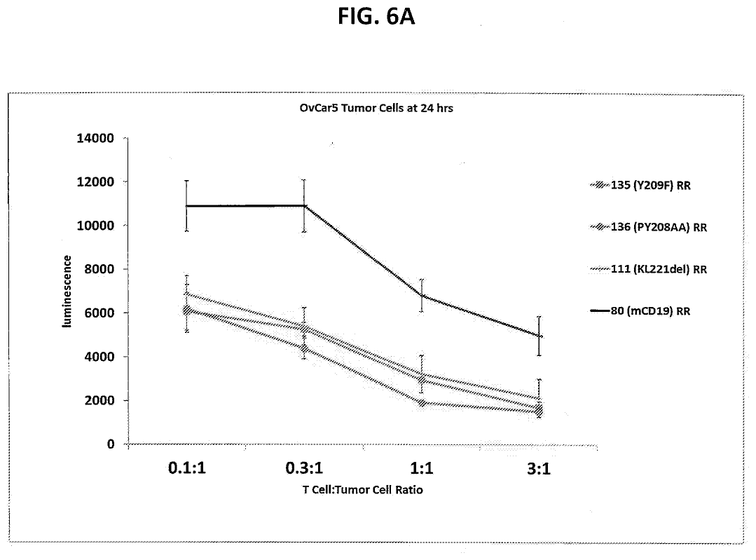

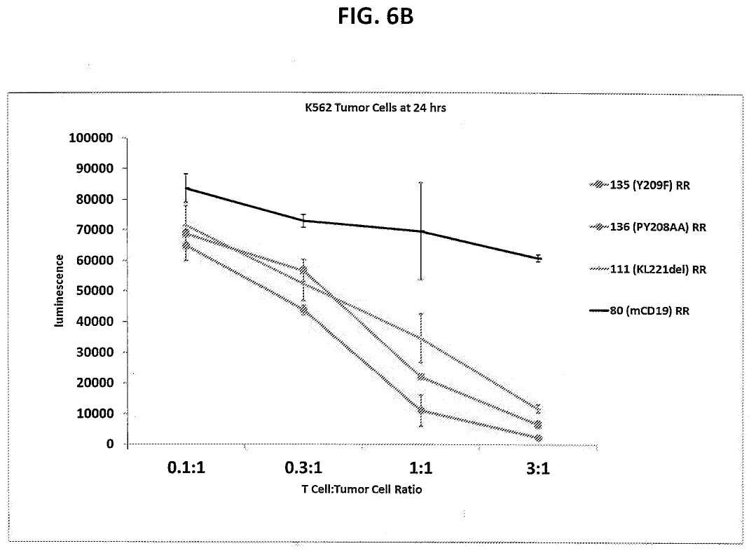

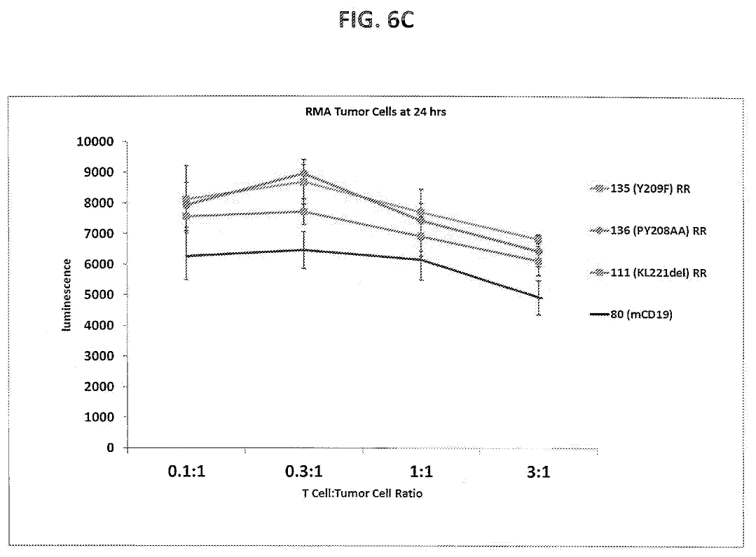

FIGS. 6A-C contain the results of cytotoxicity assays for CAR constructs JC135, JC136, and JC111. FIG. 6A contains the cytotoxicity results against ligand-positive OvCar5 tumor cells; FIG. 6B contains the cytotoxicity results against ligand-positive K562 tumor cells; FIG. 6C contains the cytotoxicity results against ligand-negative RMA tumor cells.

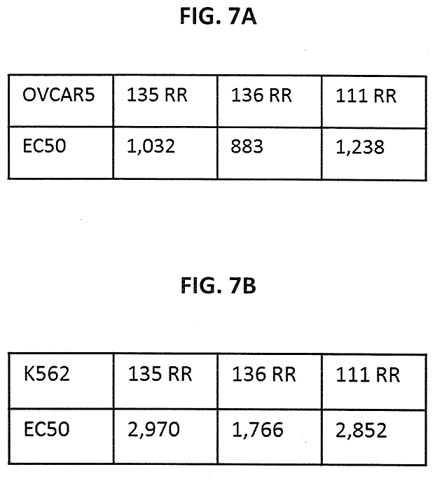

FIGS. 7A-B contain the results of cytotoxicity assays for CAR constructs JC135, JC136, and JC111. FIG. 7A contains a table of the EC₅₀ values against ligand-positive OvCar5 tumor cells; FIG. 7B contains a table of the EC₅₀ values against ligand-positive K562 tumor cells.

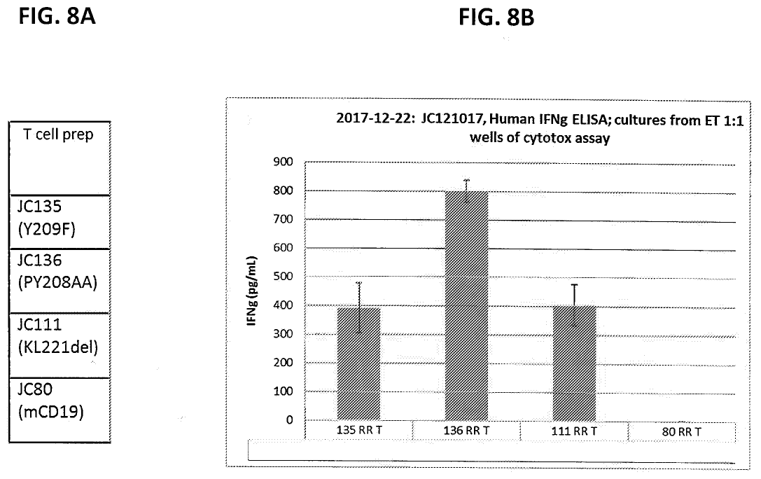

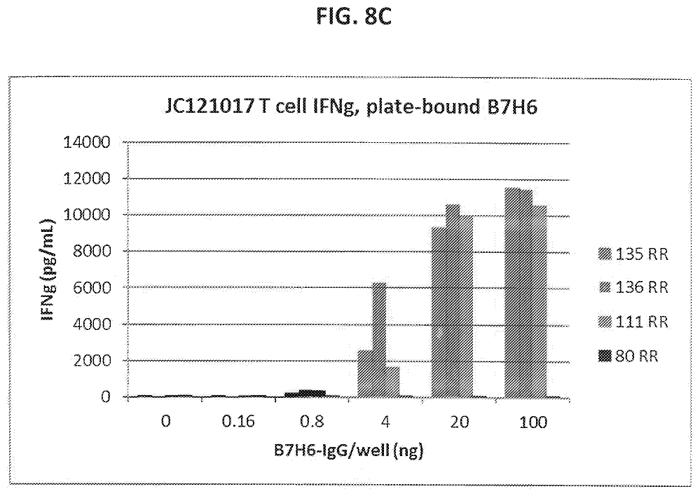

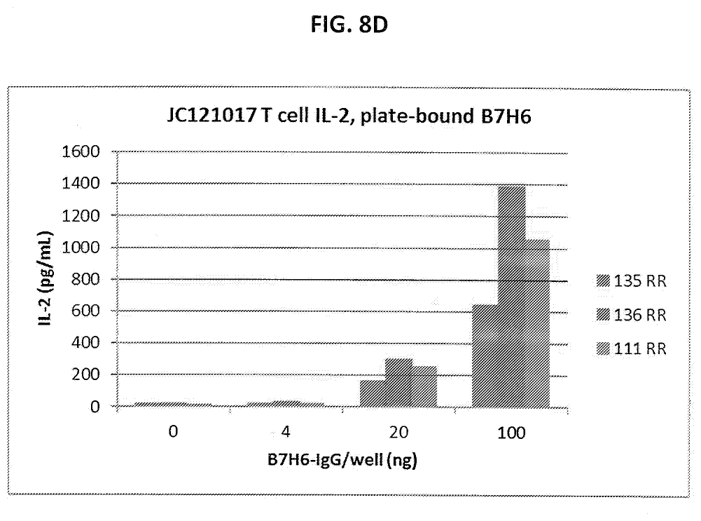

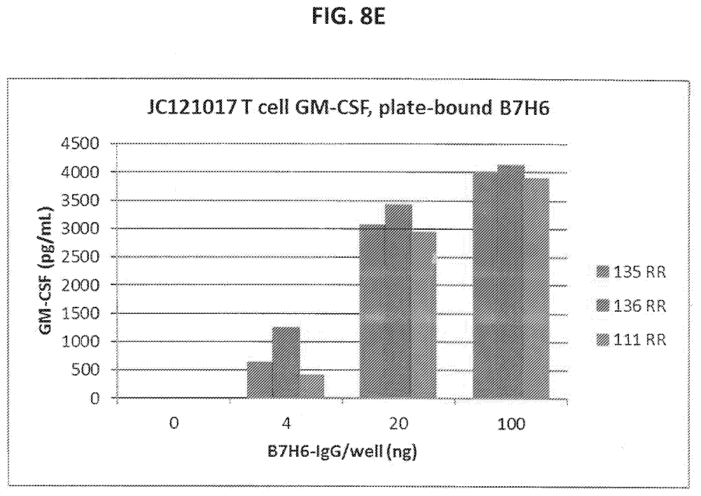

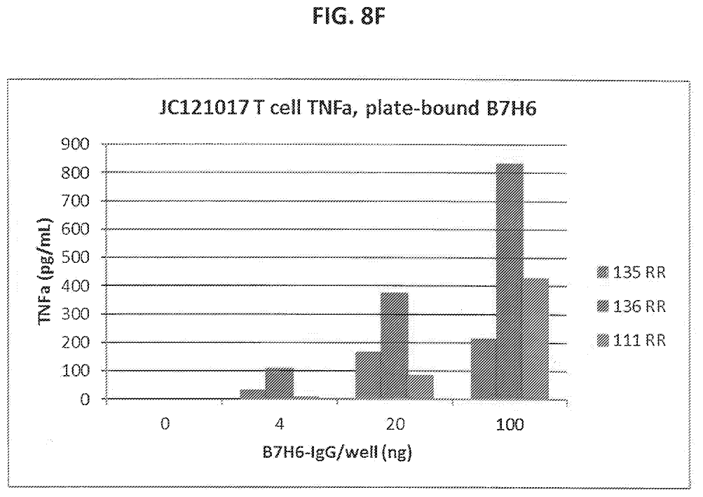

FIGS. 8A-F contain the results of cytokine production assays for CAR constructs JC135, JC136, JC111, and JC80 (mCD19 only control). FIG. 8A contains a table summarizing the T cell preparation, with descriptions of each tested construct. FIG. 8B contains a graph showing the IFNγ production for each construct in an ELISA assay at an E:T ratio of 1:1. FIG. 8C contains a graph showing the IFNγ production for constructs JC135, JC136, and JC111 at varying concentrations. FIG. 8D contains a graph showing the IL-2 production for constructs JC135, JC136, and JC111 at varying concentrations. FIG. 8E contains a graph showing the GM-CSF production for constructs JC135, JC136, and JC111 at varying concentrations. FIG. 8F contains a graph showing the TNFα production for constructs JC135, JC136, and JC111 at varying concentrations.

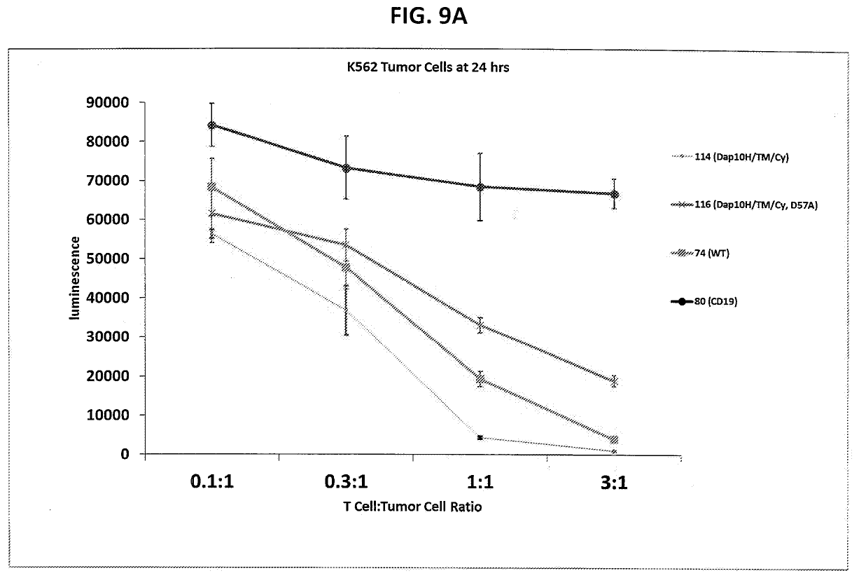

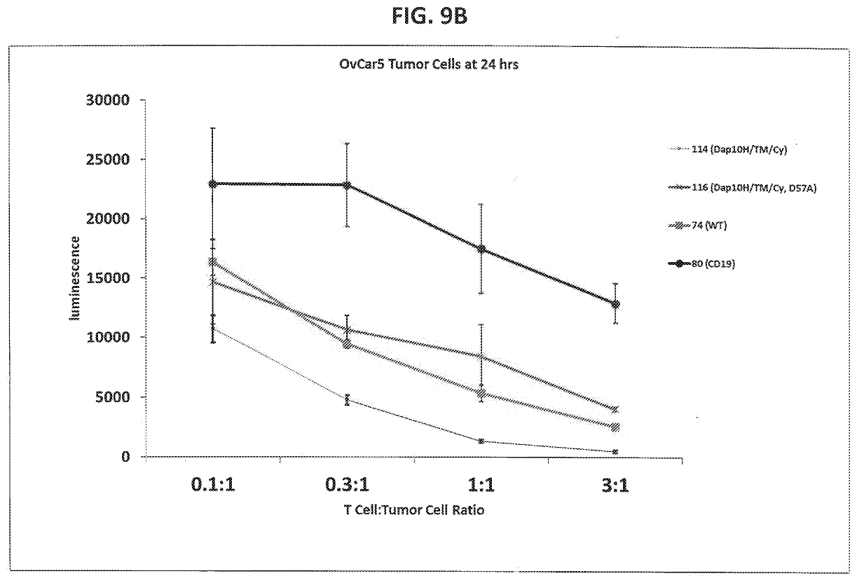

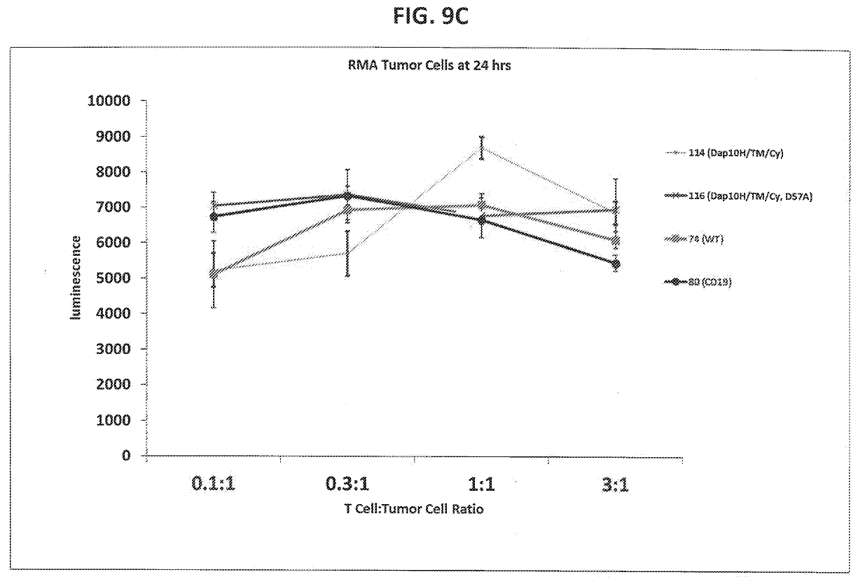

FIGS. 9A-C contain the results of cytotoxicity assays for CAR constructs JC114, JC116, JC74, and JC80 (mCD19 only control). FIG. 9A contains the cytotoxicity results against ligand-positive K562 tumor cells; FIG. 9B contains the cytotoxicity results against ligand-positive OvCar5 tumor cells; FIG. 9C contains the cytotoxicity results against ligand-negative RMA tumor cells.

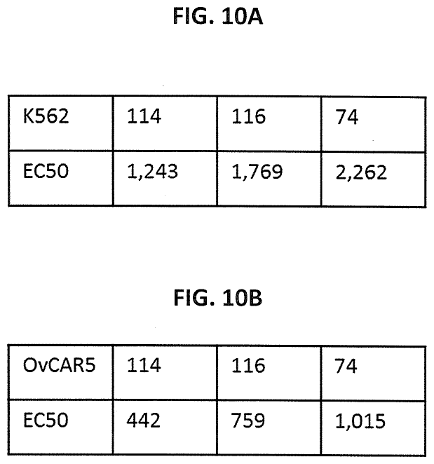

FIGS. 10A-B contain the results of cytotoxicity assays for CAR constructs JC114, JC116, and JC74. FIG. 10A contains a table of the EC₅₀ values against ligand-positive K562 tumor cells; FIG. 10B contains a table of the EC₅₀ values against ligand-positive OvCAR5 tumor cells.

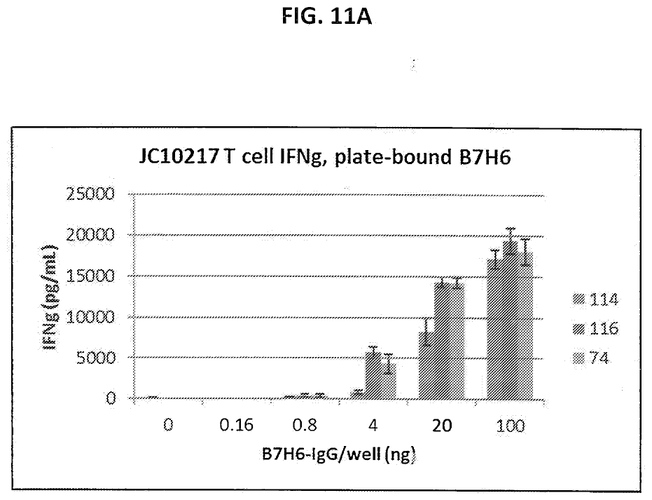

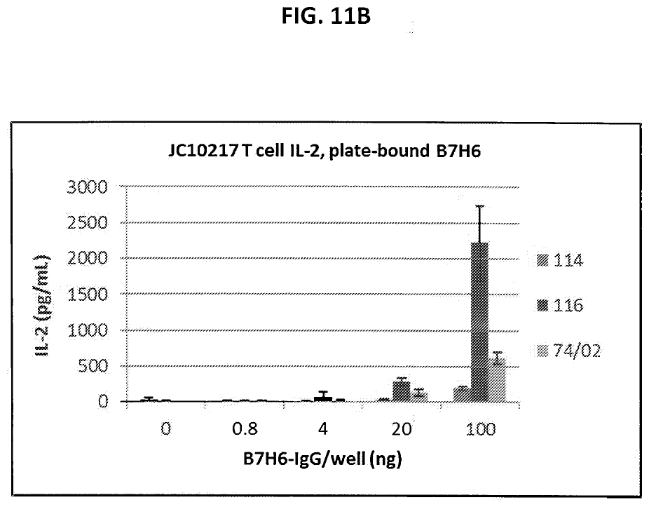

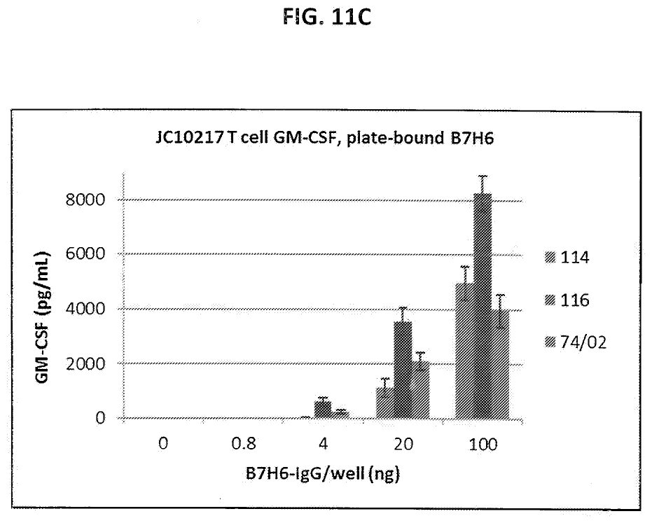

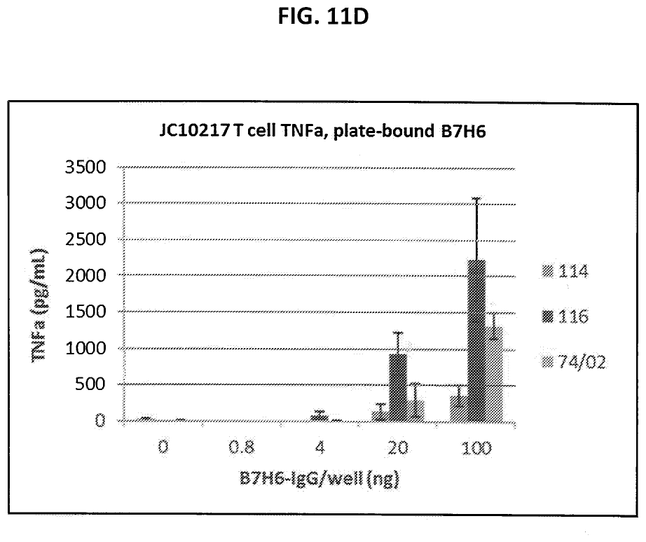

FIGS. 11A-D contain the results of cytokine production assays for CAR constructs JC114, JC116, and JC74/02. FIG. 11A contains a graph showing the IFNγ production for constructs; FIG. 11B contains a graph showing the IL-2 production; FIG. 11C contains a graph showing the GM-CSF production; FIG. 11D contains a graph showing the TNFα.

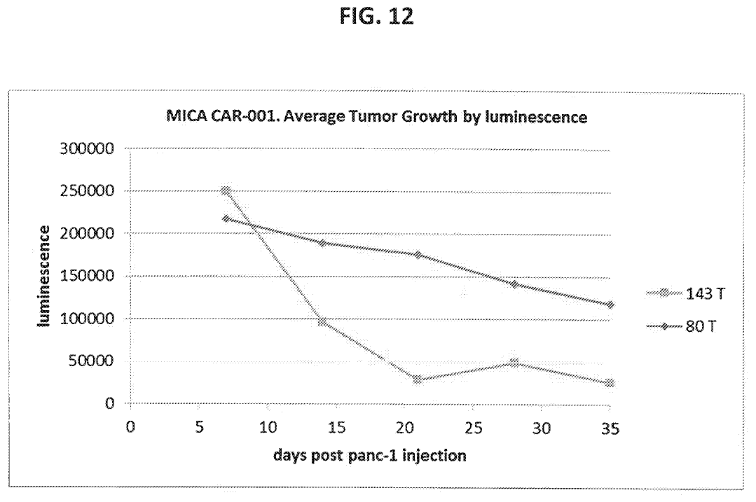

FIG. 12 contains a graph depicting the results of a tumor growth assay in NSG mice with PANC-1 expressing, luciferase-expressing tumor cells who received MICA-specific CART cells (JC143: anti-MICA CAR B2. D10h/D10D57ATM/D10cyto.Z) or control transduced T cells (JC80: mCD19 only vector) on day 12 and day 26.

### DETAILED DESCRIPTION OF THE INVENTION

### Mutations

The invention is particularly directed to novel signaling platforms for chimeric antigen receptors (CARs) and immune cells comprising such CARs. In particular, CARs of the invention may comprise one or more mutations that may alter the response of a T, NK or other immune cell comprising such CARs. In some instances, it may be beneficial to increase or decrease the immune system response in the presence of CAR-expressing T or other CAR-expressing immune cells. The mutations described herein may be used to that effect. The disclosed mutations may also be used in order to manipulate the expression of a set of cytokines by a CAR T or other immune cell or immune cells, e.g., T cells, in the vicinity of such CAR T or other CAR-expressing immune cell. Cytokines are known to be involved and in some cases essential for CAR T cell activity (Zhang, Can Res. 2007; Barber, J Immunol, 2008; Chmielewski, Can Res, 2011; Konero, Oncoimmunology, 2015). Such mutations may also be employed to modulate the activity of a T cell or other immune cell, e.g., other immune cells, e.g., T cells, in the vicinity of such CAR T cells or other CAR-expressing immune cell or in order to alter another functional feature of the CAR, CAR immune cell, or CAR T cell.

In one aspect, the invention provides genetically modified T cells that will recognize an antigen through the binding domain of a CAR and signal effector responses from the T cell. In some embodiments, the CAR may comprise specific modifications of one or more signaling domains, thus providing differential cytokine expression or other effector responses from the CAR T cells. In some embodiments, such modifications may modulate cytokine production, thus altering the local tumor microenvironment. In some embodiments, altered CAR constructs of the invention may provide control over one or more effector responses to occur. These effector responses may thus be adapted to the particular needs of the treatment or target.

In one aspect of the invention, the inventive CARs may be expressed in human T cells. In some embodiments, these CARs will trigger differential effector responses within the T cell, including, e.g., cytokine production, in the presence of their specific antigen. In some embodiments, the amounts and types of cytokines produced by a T cell comprising a CAR comprising one or more mutations as disclosed herein may differ as compared to a T cell comprising a CAR without those mutations.

In some embodiments, the CAR specificities may be combined with different modified signaling domains to activate different or modify conventional effector pathways/survival pathways in CAR immune cells, e.g., CAR T cells.

In some embodiments, a mutation to the nucleic acid sequence or amino acid sequence of a costimulation signaling domain as disclosed herein may result in altered effector responses. In some embodiments, altered effector responses may comprise lower or higher production of cytokines and/or cytotoxicity. A CAR comprising any scFv or receptor or antigen-binding domain may be modified as described herein in order to produce CAR immune cells, e.g., CAR T cells or CAR NK cells, with altered effector responses. In some embodiments, altered responses may promote specific immune activity. In some embodiments, altered responses may reduce potential toxicity.

In some embodiments, a mutation may be in one or more co-stimulatory domains. In some embodiments, a mutation may be in a hinge domain. In some embodiments, a mutation may be in a transmembrane domain. In some embodiments, a mutation may be in a cytoplasmic domain.

These mutations may alter various characteristics of the CARs or the cells comprising such CARs. In some embodiments, these mutations may improve intracellular signaling from the co-stimulatory domain. In some embodiments, the mutations may decrease intracellular signaling from the co-stimulatory domain. In some embodiments, a mutation to a CAR of the invention may reduce dimerization or interaction with other protein partners. In other embodiments, a mutation to a CAR of the invention may increase dimerization. Mutations as described herein may be used to increase or decrease binding to other proteins. Such proteins may include other CARs, PI3K, Grb2, Gads, Itk, LCK-PKCθ, FilA, ubiquitin, and/or NKG2D.

In some embodiments, a CAR construct of the invention may comprise a mutation in motif 3 of CD28. In some embodiments, a CAR construct of the invention may comprise a mutation leading to decreased interaction with Lck. In some embodiments, a decreased interaction with Lck may decrease immunosuppression by Tregs.

In some embodiments, a CAR construct of the invention may comprise a mutation that alters CAR expression within T cells. In some embodiments, the mutation may lead to increased CAR expression. In other embodiments, the mutation may lead to decreased CAR expression.

Such mutations may affect the expression of one or more cytokines. In some embodiments, a mutation to a co-stimulatory domain as described herein may increase the production of one or more cytokines. In some embodiments, a mutation to a co-stimulatory domain as described herein may decrease the production of one or more cytokines. Examples of cytokines that may be modulated include, but are not limited to, IL-1, IL-2, IL-4, IL-7, IL-9, IL-13, IL-15, IL-3, IL-5, granulocyte macrophage colony-stimulating factor (GM-CSF), IL-6, IL-11, IL-12, G-CSF, leukemia inhibitory factor (LIF), IL-10, IL-20, IL-14, IFN-α, IFN-β, IFN-γ, TNF, CD154, LT-13, TNF-α, TNF-β, 4-1BBL, APRIL, CD70, CD153, CD178, GITRL, LIGHT, OX40L, TALL-1, TRAIL, TWEAK, TRANCE, TGF-β, Epo, Tpo, Flt-3L, Stem Cell Factor (SCF), M-CSF, and MSP. In some embodiments, a mutation may affect the production of IFNγ. In some embodiments, a mutation may affect the production of IL-2. In some embodiments, a mutation may affect the production of GM-CSF. In some embodiments, a mutation may affect the production of TNFα.

Such mutations may affect cytotoxicity. In some embodiments, a CAR comprising one or more mutations as described herein may exhibit increased cytotoxicity against target cells. In some embodiments, a CAR comprising one or more mutations as described herein may exhibit decreased cytotoxicity against target cells. Some mutations may affect the specificity of the CAR T cell response.

Any one or more mutations may be combined. Alternatively, a CAR may feature only one mutation. In some embodiments, a CAR of the invention may comprise a mutation that only produces a measurable or significant change to one parameter, while other features are unaffected. For example, a CAR T cell of the invention may comprise a mutation resulting in altered IFN-γ expression, but having otherwise unchanged cytokine expression.

CD28

In some embodiments, a CAR construct of the invention may comprise one or more CD28 sequences. In a preferred embodiment, a CAR construct may comprise a CD28 co-stimulatory domain. The CAR construct may include any one or more of a CD28 hinge domain, a CD28 transmembrane domain, and a CD28 cytoplasmic domain, or any part of any one of the foregoing. In some embodiments, the CAR construct may comprise a wild type (WT) or unmodified CD28 sequence of any one or more of the hinge, transmembrane, or cytoplasmic domains. In some embodiments, the CAR construct may comprise modified CD28 sequences. Some exemplary CD28 modifications for use in the CAR constructs may include modifications to motif 1, modifications to motif 2, modifications to motif 3, modifications to the HindIII restriction site (i.e., inclusion or removal of amino acids translated from HindIII restriction site or from other such restriction enzymes), and/or modifications to the dimerization motif.

In some embodiments, the CAR construct may comprise a D190E mutation in motif 1.

In some embodiments, the CAR construct may comprise a Y191A mutation in motif 1.

In some embodiments, the CAR construct may comprise a P196A mutation in motif 2.

In some embodiments, the CAR construct may comprise a R197A mutation in motif 2.

In some embodiments, the CAR construct may comprise a Y209F mutation in motif 3.

In some embodiments, the CAR construct may comprise a PY208AA mutation in motif 3.

In some embodiments, the CAR construct may comprise a PYAPP208AYAAA mutation in motif 3.

In some embodiments, the CAR construct may comprise a KL221 deletion in the HindIII site. In some embodiments, a removal of the lysine (K) residue at this position may improve protein stability.

In some embodiments, the CAR construct may comprise a C141S mutation in the dimerization motif.

In some embodiments, a CAR construct according to the invention may comprise a CD28 co-stimulatory domain comprising any one or more of the foregoing mutations.

In other embodiments, a CAR construct of the invention may comprise one or more CD28 domains comprising further modifications, additions, deletions, and/or substitutions.

DAP10

In some embodiments, a CAR construct may comprise one or more DAP10 sequences. In a preferred embodiment, a CAR construct may comprise a DAP10 co-stimulatory domain. The CAR construct may include any one or more of a DAP10 hinge domain, a DAP10 transmembrane domain, and a DAP10 cytoplasmic domain, or any part of any one of the foregoing. In some embodiments, the CAR construct may comprise a wild type (WT) or unmodified DAP10 sequence of any one or more of the hinge, transmembrane, or cytoplasmic domains. In some embodiments, the CAR construct may comprise modified DAP10 sequences. Some exemplary DAP10 modifications for use in the CAR constructs may include modifications to the NKG2D binding motif.

In some embodiments, the CAR construct may comprise a D57A mutation in the NKG2D binding motif.

In some embodiments, a CAR construct according to the invention may comprise one or more DAP10 domains comprising one or more further modifications, additions, deletions, and/or substitutions.

### Nucleic Acid Constructs and Chimeric Antigen Receptors (CARs) Encoded Thereby

Disclosed are nucleic acid constructs that comprise genes encoding a chimeric antigen receptor (CAR). Such nucleic acid constructs can be transduced into immune cells to create an immune cell that expresses the CAR. The CAR may comprise sequences that have been mutated to alter one or more CAR features. Such properties may be beneficial in T-cell-based or an immune-cell-based immunotherapy. Thus, disclosed herein are nucleic acid constructs useful in immune cell-based immunotherapy, such as, for instance, adoptive cell transfer, and the like. Disclosed herein are the nucleic acid constructs encoding the CAR molecules, vectors comprising the same, recombinant cells comprising the same, kits and compositions comprising the same, and methods of use thereof.

As set forth in the Examples, a CAR of the invention may comprise a variety of mutations that may affect cytotoxicity and cytokine production, in particular. In some embodiments, these mutations may lead to increased cytotoxicity. In some embodiments, these mutations may lead to decreased cytotoxicity. In some embodiments, these mutations may lead to increased amounts of one or more cytokines. In some embodiments, these mutations may lead to decreased amounts of one or more cytokines. These altered functional features of CARs and CAR T cells may improve efficacy for the treatment of disease.

Therefore, disclosed are nucleic acid constructs, vectors, and immune host cells that harbor nucleic acids encoding a CAR. For the purposes of this invention, “nucleic acids” refer to single or double stranded nucleic acid molecules, which are isolated and provided in the form of RNA, a complementary polynucleotide (cDNA), a genomic polynucleotide and/or a composite polynucleotide (e.g., a combination of the above). As used herein, the term “nucleic acid construct” refers to a nucleic acid molecule, which includes nucleic acids encoding a CAR and nucleic acids encoding a transcription factor that mediates cell differentiation resulting in proinflammatory cytokine expression. In some embodiments, the nucleic acid construct is a linear naked molecule or a vector, e.g., a plasmid, a bacmid, a phagemid, a cosmid, a phage, a virus or an artificial chromosome.

In accordance with the present invention, the nucleic acid construct is transformed, transduced, transfected or otherwise introduced into a T cell or an immune cell and is transcribed and translated to produce a product (e.g., a chimeric receptor). Thus, the nucleic acid construct further includes at least one or more elements encoding, for example, a promoter for directing transcription of the CAR and transcription factor. According to some embodiments, nucleic acids encoding the CAR are operably linked to at least one promoter sequence. A coding nucleic acid is “operably linked” to a regulatory sequence (e.g., promoter) if the regulatory sequence is capable of exerting a regulatory effect on the coding sequence linked thereto. The nucleic acid encoding the CAR can be controlled by the same promoter, or by a promoter different from the promoter that controls expression of any other gene co-expressed with the CAR. Other elements that the nucleic acid construct can encode include other regulatory elements, such as a transcription enhancer; a self-cleaving peptide located between the CAR and other nucleic acid sequences; a nucleic acid encoding a protein that is capable of triggering cell suicide or elimination; one or more internal ribosomal entry sites; a gene encoding a protein whose expression allows for selection of a cell comprising the vector; and/or one or more cis-acting hydrolase elements.

A nucleic acid construct according to the present invention can be produced by any means known in the art. Nucleic acids encoding the CAR can be prepared and assembled into a complete coding sequence by standard techniques of molecular cloning (genomic library screening, PCR, primer-assisted ligation, site-directed mutagenesis, etc.). Nucleic acids encoding the other moieties (e.g., transcription factor, IRES or CHYSEL) may be similarly prepared. The resulting nucleic acids are preferably inserted into an expression vector and used to transform suitable mammalian host cells, preferably T lymphocyte cells as described herein, as well as other immune cells such as NK cells and LAK cells, and stem cells or other progenitor cells that differentiate into these T lymphocyte cells. Such nucleic acids may also be inserted into other types of immune cells such as NKT cells, γδ-T cells, monocytes, macrophages, dendritic cells, neutrophils, basophils, eosinophils, and mast cells, or immune-like cells.

The chimeric antigen receptor, also known as a CAR, artificial T cell receptor, chimeric T cell receptor, or chimeric immunoreceptor expressed by a construct according to the invention will generally comprise a fusion protein composed of an antigen targeting domain or recognition domain attached to an extracellular spacer/hinge domain, a transmembrane region that anchors the antigen targeting domain to the cell surface, and at least one signaling endodomain or cytoplasmic domain.

### Antigen Binding Domain

The terms “antigen targeting domain”, “antigen binding domain”, and “antigen recognition domain” are used interchangeably herein. Antigen targeting or antigen recognition by CAR molecules most commonly involves the use of a single chain variable fragment (scFv) that has been assembled from a monoclonal antibody. However, alternative targeting moieties include ligands (Altenschmidt, et al. (1996) Clin. Cancer Res. 2:1001-8; Muniappan, et al. (2000) Cancer Gene Ther. 7:128-134), peptides (Pameijer, et al. (2007) Cancer Gene Ther. 14:91-97), chimeric ligands (Davies, et al. (2012) Mol. Med. 18:565-576), receptor derivatives (Scholler, et al. (2012) Sci. Translation. Med. 4:Article IDS 132ra53; Zhang, et al. (2012)J. Immunol. 189:2290-9), and single domain antibodies (Sharifzadeh, et al. (2012) Cancer Res. 72:1844-52). Any desired antibody or antibody fragment thereof that specifically recognizes and binds a target antigen, such as a tumor antigen, may be incorporated in a CAR according to the invention. Antigens that are commonly expressed by diverse solid and hematological malignancies and have been shown to be amenable to CAR-directed targeting include proteins, carbohydrates, gangliosides, and the like. In particular, such molecules include CD19 (Brentjens, et al. (2007) Clin. Can. Res. 13:5426-5435; Loskog, et al. (2006) Leukemia 20:1819-1828; Brentjens, et al. (2003) Nat. Med. 9:279-286; Kochenderfer, et al. (2009) J. Immunol. 32:689-702; Cooper, et al. (2003) Blood 101:1637-44), CD20 (Wang, et al. (2004) Mol. Ther. 9:577-86), CD22 (James, et al. (2008)J. Immunol. 180:7028-7038), k light chain (Vera, et al. (2006) Blood 108:3890-7), CD38 (Mihara, et al. (2010) Br. J. Haematol. 151:37-46; Mihara, et al. (2009) J. Immunother. 32:737-43), and receptor-tyrosine-kinase-like orphan receptor 1 (ROR1) for treating B cell malignancies (Hudecek, et al. (2010) Blood 116:4532-41); CD30 for treating Hodgkin's and non-Hodgkin's lymphomas (Di Stasi, et al. (2009) Blood 113:6392-6402; Savoldo, et al. (2007) Blood 110:2620-30); CD33 (Dutour, et al. (2012) Adv. Hematol. Article ID 683065) or CD123 (Thokala, et al. (2011) Blood 118, abstract 1908) for treating myeloid malignancies; epithelial glycoprotein (EGP) 40 for targeting colon cancer (Daly, et al. (2000) Cancer Gene Ther. 7:284-291); tumor-associated glycoprotein 72 for treating gastrointestinal cancer (Hombach, et al. (1997) Gastroenterol. 113:1163-70); prostate-specific membrane antigen (Maher, et al. (2002) Nat. Biotechnol. 20:70-75; Gong, et al. (1999) Neoplasia 1:123-127) or prostate stem cell antigen (Morgenroth, et al. (2007) Prostate 67:1121-1131) for treating prostate cancer; ganglioside (GD) 3 (Abken, et al. (2001) Rec. Results Cancer Res. 158:249-264), high molecular weight melanoma-associated antigen (Westwood, et al. (2005) Proc. Natl. Acad. Sci. USA 102:19051-19056) or HLA-A1 MAGE A1 (Willemsen, et al. (2005) J. Immunol. 174:7853-8) for treating melanoma; ErbB2 (Altenschmidt, et al. (1996) Clin. Cancer Res. 2:1001-8) or mucin (MUC) 1 (Wilkie, et al. (2008) J. Immunol. 180:4901-9) for treating breast cancer; MUC1 (Wilkie, et al. (2008) J. Immunol. 180:4901-9), MUC16 (Chekmasova, et al. (2010) Clin. Cancer Res. 16:3594-3606), folate receptor-α for treating ovarian cancer (Hwu, et al. (1995) Cancer Res. 55:3369-73; Kershaw, et al. (2002) Nat. Biotechnol. 20:1221-7), CD44v7/8 for treating cervical cancer (Dall, et al. (2005) Cancer Immunol. Immunother. 54:51-60); carbonic anhydrase 9 (Weijtens, et al. (1996) J. Immunol. 157:836-43) or G250/CAIX (Lamers, et al. (2006) J. Clin. Oncol. 24:e20-22) for treating renal cell carcinoma; GD2 (Krause, et al. (1998) J. Exp. Med. 188:619-26; Rossig, et al. (2001) Int. J. Cancer 94:228-236; Kailayangiri, et al. (2012) Br. J. Cancer 106:1123-33), CD171 (Park, et al. (2007) Mol. Ther. 15:825-33) or nerve cell adhesion molecule (Gilham, et al. (2002)J. Immunol. 25:139-51) for treating neuroblastoma; Foetal acetylcholine receptor for treating rhabdomyosarcoma (Gattenlohner, et al. (2006) Cancer Res. 66:24-28); or ErB3/4 (Altenschmidt, et al. (1996) Clin. Cancer Res. 2:1001-8; Muniappan, et al. (2000) Cancer Gene Ther. 7:128-134), epidermal growth factor receptor vIII (Morgan, et al. (2012) Human Gene Ther. 23:1043-53), carcinoembryonic antigen (Haynes, et al. (2001)J. Immunol. 166:182-7; Haynes, et al. (2002) J. Immunol. 169:5780-6; Darcy, et al. (2000) J. Immunol. 164:3705-12), EGP2 (Ren-Heidenreich, et al. (2000) Human Gene Ther. 11:9-19), mesothelin (Carpenito, et al. (2009) Proc. Natl. Acad. Sci. USA 106:3360-5; Lanitis, et al. (2012) Mol. Ther. 20:633-43), natural killer group 2 member D ligands (Zhang, et al. (2005) Blood 106:1544-51), B7-H6 (Zhang, et al. (2012) J. Immunol. 189:2290-9), IL-13 receptor α2 (Kong, et al. (2012) Clin. Cancer Res. 18:5949-60; Kahlon, et al. (2004) Cancer Res. 64:9160-6; Brown, et al. (2012) Clin. Cancer Res. 18:2199-2209), Lewis Y (Westwood, et al. (2005) Proc. Natl. Acad Sci. USA 102:19051-6), HLA-A2 NY-ESO-1 (Schuberth, et al. (2013) Gene Ther. 20:386-95), CD44v6 (Hekele, et al. (1996) Internatl. J. Cancer 68:232-8), αᵥβ₆ integrin (Pameijer, et al. (2007) Cancer Gene Ther. 14:91-7), 8H9 (Cheung, et al. (2003) Hybrid. Hybrid. 22:209-218), vascular endothelial growth factor receptors (Niederman, et al. (2002) Proc. Natl. Acad. Sci. USA 99:7009-14; Kershaw, et al. (2000) Human Gene Ther. 11:2445-52), or 5T4 (Jiang, et al. (2006) J. Immunol. 177:4288-98) to treat a variety of cancers including, but not limited to, breast cancer, glioma, colon cancer, ovarian cancer, and multiple myeloma. In addition to antigen-specific approaches, two “universal” CAR systems have been described. These generic CARs containing avidin (Urbanska, et al. (2012) Cancer Res. 72:1844-52) or anti-fluorescein isothiocyanate (FITC) scFv (Ang, et al. (2011) Mol. Ther. 19:abstract 353; Chmielewski, et al. (2004)J. Immunol. 173:7647-7653), enabling their use in conjunction with separate targeting moieties that have been biotinylated or conjugated to FITC, respectively.

In exemplary, non-limiting embodiments, the antigen binding domain binds to B7H6. In some embodiments, the antigen binding domain binds to MICA.

In embodiments wherein the antigen targeting domain is an scFv, the scFv can be derived from the variable heavy chain (V_{H}) and variable light chain (V_{L}) regions of an antigen-specific mAb linked by a flexible linker. The scFv retains the same specificity and a similar affinity as the full antibody from which it was derived (Muniappan, et al. (2000) Cancer Gene Ther. 7:128-134). Various methods for preparing an scFv can be used including methods described in U.S. Pat. No. 4,694,778; Bird, et al. (1988) Science 242:423-442; Ward, et al. (1989) Nature 334:54454; and Skerra, et al. (1988) Science 242:1038-1041. In certain embodiments, the scFv is humanized or is a fully human scFv.

Non-scFv antigen targeting domains include, e.g., the CD27 receptor (Shaffer, et al. (2011) Blood 117:4304-4314), the heregulin molecule (a ligand for Her3 and Her4 receptors) (Muniappan, et al. (2000) Cancer Gene Ther. 7:128), interleukin (IL)-13 mutein (Kahlon, et al. (2004) Cancer Res. 64:9160-6), vascular endothelial growth factor (anti-VEGFR2) (Niedeman, et al. (2002) Proc. Natl. Acad. Sci. USA 99:7009-14), a chimeric NKp30 CAR targeting B7-H6 (NKp30 ligand) (Zhang, et al. (2012) J. Immunol. 189:2290-2299), variable regions of a T cell receptor (e.g., TCRα, TCRβ, TCRγ, or TCR δ), CD8α, CD8β, CD11A, CD11B, CD11C, CD18, CD29, CD49A, CD49B, CD49D, CD49E, CD49F, CD61, CD41, and CD51.

In some embodiments, the CAR of the invention may target antigens of the B7 family, such as B7-H6. NKp30 recognizes B7-H6 and BAT-3 (Brandt et al., 2009; Pogge von Strandmann et al., 2007). B7-H6 is a member of the B7 family (which includes ligands for stimulatory/inhibitory T cell co-receptors CD28/CTLA4) and is poorly expressed on normal cells, but up-regulated in different tumor cell lines. (See, Vitale, C., Mingari, M. C., Vitale, M., Balsamo, M. and Zambello, R., 2012, Physiological and Pathological Aspects of Human NK Cells. INTECH Open Access Publisher).

Other specific examples of targeting domains include C-type lectin-like NK cell receptors that bind MIC-A, MIC-B, heat shock proteins, ULBP binding proteins (e.g., ULPBs 1-4), and non-classical HLA molecules such as HLA-E and HLA-G. Exemplary NK cell receptors of this type include, but are not limited to, Dectin-1 (GENBANK accession number AJ312373 or AJ312372), Mast cell function-associated antigen (GENBANK accession number AF097358), HNKR-P1A (GENBANK accession number U11276), LLT1 (GENBANK accession number AF133299), CD69 (GENBANK accession number NM_001781), CD69 homolog, CD72 (GENBANK accession number NM_001782), CD94 (GENBANK accession number NM_002262 or NM_007334), KLRF1 (GENBANK accession number NM_016523), Oxidised LDL receptor (GENBANK accession number NM_002543), CLEC-1, CLEC-2 (GENBANK accession number NM_016509), NKG2D (GENBANK accession number BC039836; Zhang, et al. (2006) Cancer Res. 66:5927-5933; Song, et al. (2013) Hum. Gene Ther. 24:295-305; Lehner, et al. (2012) PLoS One 7:e31210; U.S. Pat. No. 7,994,298), NKG2C (GENBANK accession number AJ001684), NKG2A (GENBANK accession number AF461812), NKG2E (GENBANK accession number AF461157), WUGSC:H_DJ0701016.2, or Myeloid DAP12-associating lectin (MDL-1; GENBANK accession number AJ271684). Similar type I receptors, which can be used in the CAR of this invention, include NKp46 (e.g., GENBANK accession number AJ001383), NKp30 (e.g., GENBANK accession number AB055881), or NKp44 (e.g., GENBANK accession number AJ225109).

A protein associated with a C-type lectin-like NK cell receptor protein can also be used in the CAR of the invention. In general, proteins associated with C-type lectin-like NK cell receptor are defined as proteins that interact with the receptor and transduce signals therefrom. Suitable human proteins that function in this manner include, but are not limited to DAP10 (e.g., GENBANK accession number: AAH65224.1; U.S. Pat. No. 8,252,914) and DAP12 (e.g., GENBANK accession number AF019562).

A CAR of the invention can also include an antigen targeting domain capable of binding to an antigen derived from Retroviridae (e.g., human immunodeficiency viruses such as HIV-1 and HIV-LP), Picornaviridae (e.g., poliovirus, hepatitis A virus, enterovirus, human coxsackievirus, rhinovirus, and echovirus), rubella virus, coronavirus, vesicular stomatitis virus, rabies virus, Ebola virus, parainfluenza virus, mumps virus, measles virus, respiratory syncytial virus, influenza virus, hepatitis B virus, parvovirus, Adenoviridae, Herpesviridae (e.g., type 1 and type 2 herpes simplex virus (HSV), varicella-zoster virus, cytomegalovirus (CMV), and herpes virus), Poxviridae (e.g., smallpox virus, vaccinia virus, and pox virus), or hepatitis C virus. For example, CARs for the treatment of hepatitis C virus, hepatitis B virus and influenza virus have been described. See, e.g., Sautto, et al. (2015) Gut doi:10.1136/gutjnl-2014-308316; Krebs, et al. (2013) Gastroenterol. 145:456-65; and Talbot, et al. (2013) Open Virol. J. 7:28-36.

Further, a CAR of the invention can also include an antigen binding domain that binds to an antigen derived from a bacterial strain of Staphylococci, Streptococcus, Escherichia coli, Pseudomonas, or Salmonella. Particularly, there is provided a CAR capable of binding to an antigen derived from an infectious bacterium, for example, Helicobacter pyloris, Legionella pneumophilia, a bacterial strain of Mycobacteria sps. (e.g., M. tuberculosis, M. avium, M. intracellulare, M. kansasii, or M. gordonea), Staphylococcus aureus, Neisseria gonorrhoeae, Neisseria meningitides, Listeria monocytogenes, Streptococcus pyogenes, Group A Streptococcus, Group B Streptococcus (Streptococcus agalactiae), Streptococcus pneumoniae, or Clostridium tetani.

Further, a CAR of the invention can also include an antigen binding domain the binds to an autoantigen or self-antigen, an allergen, or an antigen or receptor expressed by cells involved in triggering autoimmunity or inflammation.

The antigen binding domain may be derived from a polypeptide that binds to a target antigen. In some embodiments, the polypeptide may be a receptor or a portion of a receptor that binds to an antigen. In another embodiment, the antigen binding domain may be derived from ligands that bind to an antigen.

In another embodiment, the antigen binding domain may be derived from an antibody or antigen binding fragment thereof that binds to an antigen. Examples of antibody fragments include, but are not limited to, fragment antigen binding (Fab) fragments, F(ab′)₂ fragments, Fab′ fragments, Fv fragments, recombinant IgG (rIgG) fragments, single chain antibody fragments, single chain variable fragments (scFv), single domain antibodies (e.g., sdAb, sdFv, nanobody) fragments, diabodies, and multi-specific antibodies formed from antibody fragments. In particular embodiments, the antibodies are single-chain antibody fragments comprising a variable heavy chain region and/or a variable light chain region, such as scFvs.

Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that may not be produced by enzyme digestion of a naturally-occurring intact antibody. In some aspects, the antibody fragments are scFvs.

In some aspects, the antigen binding domain may be derived from an antibody or antigen-binding fragment thereof that has one or more specified functional features, such as binding properties, including binding to particular epitopes, such as epitopes that are similar to or overlap with those of other antibodies, the ability to compete for binding with other antibodies, and/or particular binding affinities.

In some embodiments, the antigen binding domain binds to an epitope containing one or more amino acids within (or is entirely within) an extracellular domain of a target antigen and/or within (or is entirely within) a membrane-proximal region of the extracellular portion of a target antigen.

In some embodiments, the antigen binding domain, the CARs comprising such, and the cells comprising such CARs display a binding preference for target antigen-expressing cells as compared to target antigen-negative cells. In some embodiments, the binding preference is observed where a significantly greater degree of binding is measured to the antigen-expressing, as compared to the non-expressing, cells. In some cases, the total degree of observed binding to the target antigen or to the antigen-expressing cells is approximately the same, at least as great, or greater than that observed for non-antigen specific domains, CARs, or cells. In any of the provided embodiments, comparison of binding properties, such as affinities or competition, may be via measurement by the same or similar assay.

In some embodiments, the antigen binding domain comprises an scFv comprising the CDR sequences of a target antigen binding antibody. CDRs may be determined using conventional methods, The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745.” (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme).

In an embodiment, the sequence comprising the antigen binding domain further comprises a leader sequence or signal sequence. In embodiments where the antigen binding domain comprises an scFv, the leader sequence may be positioned at the amino terminus of the scFv. In some embodiments, when the heavy chain variable region is N-terminal, the leader sequence may be positioned at the amino terminus of the heavy chain variable region. In some embodiments, when the light chain variable region is N-terminal, the leader sequence may be positioned at the amino terminus of the light chain variable region. The leader sequence may comprise any suitable leader sequence.

### Hinge

In some embodiments, the CAR comprises a linker, spacer, or hinge sequence between the antigen binding domain and the transmembrane domain and/or between the transmembrane domain and the cytoplasmic domain. A linker, spacer, or hinge refers to any oligopeptide or polypeptide that serves to link the transmembrane domain with the antigen targeting domain and/or the transmembrane domain with the intracellular signaling endodomain. The spacer domain can be up to 300 amino acids, preferably 10 to 100 amino acids, 25 to 50 amino acids or 2 to 10 amino acids in length. One of ordinary skill in the art will appreciate that a hinge sequence is a short sequence of amino acids that facilitates flexibility (see, e.g., Woof et al., Nat. Rev. Immunol., 4(2): 89-99 (2004)). The hinge sequence can be any suitable sequence derived or obtained from any suitable molecule. In some embodiments, the length of the hinge sequence may be optimized based on the distance between the CAR and the binding epitope, e.g., longer hinges may be optimal for membrane proximal epitopes.

In some embodiments, the CAR, such as the antigen binding portion thereof, further includes a hinge, linker or spacer. In preferred embodiments, the hinge, linker or spacer may be derived from CD8, CD28, or DAP10. In some embodiments, the hinge region may comprise a sequence allowing for dimerization. In some embodiments, the hinge region may comprise a mutation that decreases dimerization. In some embodiments, the hinge region may comprise a mutation that prevents dimerization.

In some embodiments, the hinge may be derived from or include at least a portion of an immunoglobulin Fc region, for example, an IgG1 Fc region, an IgG2 Fc region, an IgG3 Fc region, an IgG4 Fc region, an IgE Fc region, an IgM Fc region, or an IgA Fc region. In certain embodiments, the spacer domain includes at least a portion of an IgG1, an IgG2, an IgG3, an IgG4, an IgE, an IgM, or an IgA immunoglobulin Fc region that falls within its CH2 and CH3 domains. In some embodiments, the spacer domain may also include at least a portion of a corresponding immunoglobulin hinge region. In some embodiments, the hinge is derived from or includes at least a portion of a modified immunoglobulin Fc region, for example, a modified IgG1 Fc region, a modified IgG2 Fc region, a modified IgG3 Fc region, a modified IgG4 Fc region, a modified IgE Fc region, a modified IgM Fc region, or a modified IgA Fc region. The modified immunoglobulin Fc region may have one or more mutations (e.g., point mutations, insertions, deletions, duplications) resulting in one or more amino acid substitutions, modifications, or deletions that cause impaired binding of the spacer domain to an Fc receptor (FcR). In some aspects, the modified immunoglobulin Fc region may be designed with one or more mutations which result in one or more amino acid substitutions, modifications, or deletions that cause impaired binding of the spacer domain to one or more FcR including, but not limited to, FcγRI, FcγR2A, FcγR2B1, FcγR2B2, FcγR3A, FcγR3B, FcεRI, FcεR2, FcαRI, Fcα/μR, or FcRn. In some embodiments, a spacer may include an IgG4 hinge alone, IgG4 hinge linked to CH2 and CH3 domains, or IgG4 hinge linked to the CH3 domain.

In some aspects, the hinge, spacer, or linker can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include a CD28 hinge, a CD8 hinge, or a DAP10 hinge. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635.

The spacer domain preferably has a sequence that promotes binding of a CAR with an antigen and enhances signaling in a cell. Examples of an amino acid that is expected to promote the binding include cysteine, a charged amino acid, and serine and threonine in a potential glycosylation site, and these amino acids can be used as an amino acid constituting the spacer domain. Further, the spacer domain may be an artificially synthesized sequence.

### Transmembrane Domain

With respect to the transmembrane domain, the CAR can be designed to comprise a transmembrane domain that is fused to the antigen binding domain of the CAR. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of a receptor complex.

The transmembrane domain may be derived either from a natural or from a synthetic source. Where the source is natural, the domain may be derived from any membrane-bound or transmembrane protein. Typically, the transmembrane domain denotes a single transmembrane a helix of a transmembrane protein, also known as an integral protein. Transmembrane regions of particular use in this invention may be derived from (i.e. comprise at least the transmembrane region(s) of) CD28, CD3ε, CD4, CD5, CD8, CD9, CD16, CD22, CD33, CD37, CD45, CD64, CD80, CD86, CD134, CD137, CD154, TCRα, TCR β, H2-Kb, FcεRIγ, GITR or CD3 ζ and/or transmembrane regions containing functional variants thereof such as those retaining a substantial portion of the structural, e.g., transmembrane, properties thereof can be used. See, e.g., Kahlon, et al. (2004) Cancer Res. 64:9160-9166; Schambach, et al. (2009) Methods Mol. Biol. 506:191-205; Jensen, et al. (1998) Biol. Blood Marrow Transplant 4:75-83; Patel, et al. (1999) Gene Ther. 6:412; Song, et al. (2012) Blood 119:696-706; Carpenito, et al. (2009) Proc. Natl. Acad. Sci. USA 106:3360-5; Hombach, et al. (2012) Oncoimmunology 1:458-66) and Geiger, et al. (2001) Blood 98:2364-71.

Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. A transmembrane domain of the invention is thermodynamically stable in a membrane. It may be a single alpha helix, a transmembrane beta barrel, a beta-helix of gramicidin A, or any other structure. Transmembrane helices are usually about 20 amino acids in length.

Optionally, a short oligo- or polypeptide linker, preferably between 2 and 10 amino acids in length may form the linkage between the transmembrane domain and the intracellular signaling domain(s) of the CAR. A glycine-serine doublet may provide a suitable linker.

In some embodiments, the transmembrane domain is a CD28 transmembrane domain. In some embodiments, the transmembrane domain is a CD28 transmembrane domain comprising one or more mutations. In some embodiments, the transmembrane domain is a DAP10 transmembrane domain. In some embodiments, the transmembrane domain is a DAP10 transmembrane domain comprising one or more mutations.

### Cytoplasmic Domain/Intracellular Signaling Domain

The intracellular signaling domain or otherwise the cytoplasmic domain of the CAR of the invention triggers or elicits activation of at least one of the normal effector functions of the immune cell in which the CAR has been placed. The term “effector function” refers to a specialized function of a cell. Effector function of a T cell, for example, may be cytolytic activity or helper activity, including the secretion of cytokines. Thus, the term “intracellular signaling domain” refers to the portion of a protein which transduces the effector function signal and directs the cell to perform a specialized function(s). While usually the entire intracellular signaling domain can be employed, in many cases it is not necessary to use the entire chain. To the extent that a truncated portion of the intracellular signaling domain is used, such truncated portion may be used in place of the intact chain as long as it transduces an effector function signal. The term intracellular signaling domain is thus meant to include any truncated portion of the intracellular signaling domain sufficient to transduce an effector function signal.

In some embodiments, the intracellular signaling endodomain transmits a signal into a cell when the extracellular antigen targeting domain present within the same molecule binds to (interacts with) an antigen. Natural T cell-activation is transmitted by two different kinds of cytoplasmic signaling endodomains, that is, a sequence for initiating antigen-dependent primary activation via a TCR complex (primary cytoplasmic signaling endodomain) and a sequence for acting antigen-independently to provide a secondary or costimulatory signal (secondary cytoplasmic signaling endodomain or costimulatory endodomain). Therefore, while some embodiments embrace a CAR with only a primary cytoplasmic signaling endodomain, in other embodiments, a CAR of the invention includes a primary signaling endodomain and one or more secondary cytoplasmic signaling endodomains.

The primary cytoplasmic signaling endodomain regulates primary activation of a TCR complex. The primary cytoplasmic signaling sequence that stimulates the activation may include a signal transduction motif known as an immunoreceptor tyrosine-based activation motif (ITAM) (Asp/Glu)-Xaa-Xaa-Tyr*-Xaa-Xaa-(Ile/Leu)-Xaa₆₋₈-Tyr*-Xaa-Xaa-(Ile/Leu) (SEQ ID NO:1) (Reth, et al. (1989) Nature 338:383-384). On the other hand, the primary cytoplasmic signaling endodomain that acts in an inhibitory way includes a signal transduction motif known as an immunoreceptor tyrosine-based inhibition motif (ITIM) (Burshtyn, et al. (1999) J. Immunol. 162:897-902). In the present invention, an intracellular signaling endodomain having an ITAM or an ITIM can be used.

Examples of ITAM-containing primary cytoplasmic signaling sequences that are of particular use in the invention include those derived from an intracellular signaling domain of a lymphocyte receptor chain, a TCR/CD3 complex protein, an Fc receptor subunit, an IL-2 receptor subunit, CD3 ζ, FcR γ, FcR β, CD3 γ, CD3 δ, CD3 ε, CD5, CD22, CD79a, CD79b, CD66d, CD278 (ICOS), FcεRI, DAP10, and DAP12. It is particularly preferred that the intracellular signaling domain in the CAR of the invention comprises a cytoplasmic signaling sequence derived from CD3ζ. Specifically, examples of the ITAM include residues 51 to 164 of CD3 (GENBANK Accession No. NP 932170), residues 45 to 86 of FcεRIγ (GENBANK Accession No. NP_004097), residues 201 to 244 of FcεRIβ (GENBANK Accession No. NP_000130), residues 139 to 182 of CD3γ (GENBANK Accession No. NP_000064), residues 128 to 171 of CD3δ (GENBANK Accession No. NP 000723), residues 153 to 207 of CD3ε (GENBANK Accession No. NP 000724), residues 402 to 495 of CD5 (GENBANK Accession No. NP_055022), residues 707 to 847 of CD22 (GENBANK Accession No. NP 001762), residues 166 to 226 of CD79a (GENBANK Accession No. NP 001774), residues 182 to 229 of CD79b (GENBANK Accession No. NP_000611), and residues 177 to 252 of CD66d (GENBANK Accession No. NP_001806), and their variants having the same function as these peptides have. The referenced residues are based on amino acid sequence information from GENBANK and is based on the full length of the precursor (including a signal peptide sequence etc.) of each protein.

Preferred examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

In a preferred embodiment, the cytoplasmic domain of the CAR can be designed to comprise the CD3-ζ signaling domain by itself or combined with any other desired cytoplasmic domain(s) useful in the context of the CAR of the invention. For example, the cytoplasmic domain of the CAR can comprise a CD3 chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen.

Various co-stimulatory domains have been reported to confer differing properties. For example, the 4-1BB co-stimulatory domain showed enhanced persistence in in vivo xenograph models (Milone et al. Mol Ther 2009; 17:1453-1464; Song et al. Cancer Res 2011; 71:4617-4627). Additionally, these different co-stimulatory domains produce different cytokine profiles which, in turn, may produce effects on target cell-mediated cytotoxicity and the tumor microenvironment. Indeed, DAP10 signaling in NK cells has been associated with an increase in Th1 and inhibition of Th2 type cytokine production in CD8⁺ T cells (Barber et al. Blood 2011; 117:6571-6581).

Examples of co-stimulatory molecules include an MHC class I molecule, TNF receptor proteins, immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8α, CD8β, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, CRTAM, OX40 (CD134), 4-1BB (CD137), SLAM (SLAMF1, CD150, IPO-3), CD160 (BY55), SELPLG (CD162), DNAM1 (CD226), Ly9 (CD229), SLAMF4 (CD244, 2B4), ICOS (CD278), CEACAM1, CDS, CRTAM, DAP10, GADS, GITR, HVEM (LIGHTR), IA4, ICAM-1, IL2R β, IL2R γ, IL7Rα, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, a ligand that specifically binds with CD83, and the like.

While any suitable endodomain can be used in the CAR of the invention, in certain embodiments, the invention specifically contemplates the use of all or a part of the endodomains of CD28 and/or DAP10 and CD3. In specific embodiments, intracellular signaling endodomains are those of the T cell antigen receptor complex; e.g., CD28, DAP10, CD137, CD2, which are used either alone or in a series with CD3ζ. One or multiple endodomains may be employed, as so-called third generation CARs have at least 2 or 3 signaling domains fused together for additive or synergistic effect, for example.

The cytoplasmic signaling sequences within the intracellular signaling domain of the CAR of the invention may be linked to each other in a random or specified order. In a CAR containing more than one intracellular endodomain, an oligopeptide linker, as described above, or a polypeptide linker can be inserted between the intracellular endodomains to link the domains. Optionally, a short oligo- or polypeptide linker, preferably between 2 and 10 amino acids in length may form the linkage. A glycine-serine doublet or continuous sequence provides a particularly suitable linker.

Specific examples of secondary cytoplasmic signaling endodomains or costimulatory endodomains that can be used in the present invention include CD28 and DAP10.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.0.1% from the specified value, as such variations are appropriate to perform the disclosed methods.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are showing some response which by way of example may include these cells producing a cytokine, eliciting cytotoxicity, expressing or not expressing certain gene or genes, such as activation makers (e.g., CD69, CD25, CD44, CD45RA, and/or CD45RO) and lymphnode homing markers (e.g., CD62L and CCR7), and/or proliferating in an antigen-specific manner.

The term “adoptive cell therapy” or “adoptive T-cell therapy” or “ACT” as used herein means the transfer of cells into a patient, where the cells have been engineered to or otherwise altered prior to transfer into the subject. ACT is also referred to as adoptive T-cell immunotherapy, etc. An example of ACT is the harvesting from a subject's blood or tumor, an immune cell, such as a T cell. These immune cells are then stimulated ex vivo, in culture and expanded. The cells are then transduced with one or more nucleic acid constructs that allow the cell to express new molecules, such as a CAR, providing the engineered immune cells with a new mechanism for combating a disease, for instance a cancer. In some instances, the CAR will comprise an antigen binding domain that specifically recognizes an antigen expressed by a tumor or cancer. The CAR may recognize an antigen related to other diseases or conditions. Typical immune cells utilized in ACT procedures include tumor-infiltrating lymphocytes (TIL) or T cells. Immune cells used in ACT can be derived from the patient/subject themselves, or from a universal donor. Alternatively, an appropriate cell line such as an NK cell line (e.g., NK-92) or a desired type of immune cells (e.g., T cells, regulatory T cells, NK cells, NKT cells, γδ-T cells, macrophages, and dendritic cells) artificially generated from stem cells (e.g., hematopoietic stem cells or induced pluripotent stem cells) may also be used in ACT. ACT may also be accompanied by the optional step of lymphodepletion of the subject's own lymphocytes that may compete with the recombinant cells infused back into the subject. For example, in some embodiments, ACT may comprise harvesting autologous or allogeneic T cells and transducing these T cells with one or more nucleic acid constructs, so that the T cells express a CAR, and then infusing the cells into a subject in need thereof.

The term “allogeneic” or “donor-derived” refers to any material derived from a different animal of the same species as the individual to whom the material is introduced. Two or more individuals are said to be allogeneic to one another when the genes at one or more loci are not identical. In many cases, allogeneic material from individuals of the same species are sufficiently unlike genetically so that they interact antigenically.

The term “anti-tumor effect” or “anti-tumor cytotoxicity” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place. The term may also refer to any cytocidal activity against target tumor cells resulting from the exposure of these target cells to cells bearing the nucleic acid constructs described herein. This activity may be measured by known cytotoxicity assays, including, e.g., IFN-γ production assays and luciferase assays.

The term “antibody,” as used herein, refers to an immunoglobulin molecule which specifically binds with an antigen. For example, in one aspect, the antigen is B7-H6. In another aspect, the antigen is MICA. Antibodies can be intact immunoglobulins derived from natural sources or from recombinant sources and can be immunoreactive portions of intact immunoglobulins. The term is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (scFv), diabodies, and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific (e.g., bispecific) antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

The term “antibody fragment” refers to a portion of an intact antibody and refers to the antigenic determining variable regions of an intact antibody. Examples of antibody fragments include, but are not limited to, fragment antigen binding (Fab) fragments, F(ab′)₂ fragments, Fab′ fragments, Fv fragments, recombinant IgG (rIgG) fragments, single chain antibody fragments, including single chain variable fragments (scFv), single domain antibodies (e.g., sdAb, sdFv, nanobody) fragments, diabodies, and multispecific antibodies formed from antibody fragments. In a specific embodiment, the antibody fragment may be an scFv.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. Kappa and lambda light chains refer to the two major antibody light chain isotypes.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides or even carbohydrates, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated, synthesized, or can be derived from a biological sample, or might be macromolecule besides a polypeptide. Such a biological sample can include, but is not limited to a tissue sample, a tumor sample, a cell or a fluid with other biological components.

The term “antigen binding domain” refers to one or more extracellular domains of the chimeric antigen receptor which have specificity for a particular antigen.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “autologous” refers to any material derived from the same individual to whom it is later to be re-introduced.

The terms “B7H6”, “B7-H6”, and “NCR3LG1” refer to natural killer cell cytotoxicity receptor 3 ligand 1, a specific ligand for the NK cell-activating receptor NKp30. This protein is expressed on various types of primary human tumors, including leukemia, lymphoma, and gastrointestinal stromal tumors, but it is not known to be constitutively expressed on normal tissues. In some embodiments, the nucleic acid construct or constructs of the invention encode a CAR that specifically binds to B7H6.

The term “bind” refers to an attractive interaction between two molecules that results in a stable association for a period of time in which the molecules are in close proximity to each other. The result of molecular binding is sometimes the formation of a molecular complex in which the attractive forces holding the components together are generally non-covalent, and thus are normally energetically weaker than covalent bonds.

The term “bystander immune cells” refers to immune cells which are in the vicinity of CAR expressing immune cells according to the invention wherein at least one activity or function thereof, e.g., cytotoxicity, cytokine expression, cytokine profile or other effector function is modulated (increased or inhibited) by CARs or CAR expressing cells according to the invention.

The term “cancer” refers to a disease characterized by the uncontrolled growth of aberrant cells. Cancer cells can spread locally or through the bloodstream and lymphatic system to other parts of the body. Examples of various cancers are described herein and include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, cancer of the colon, liver, cervix, lung, pancreas, prostate, leukemia, lymphoma, gastrointestinal stromal tumor, prohemocytic leukemia, B-cell lymphoma, monocytic lymphoma, erythro leukemia, Burkitt's lymphoma, chronic myelogenous leukemia (CML), T and B lymphomas, myeloid leukemias, melanomas, carcinomas, large T SV40 antigen-transformed cells, acute nonlymphoblastic leukemia (ANLL), acute lymphoblastic leukemia (ALL), and non-Hodgkin's and Hodgkin's lymphoma, T-ALL, marginal zone lymphoma, and the like.

The term “CD28” refers to the protein Cluster of Differentiation 28, one of the proteins expressed on T cells that provide co-stimulatory signals required for T cell activation and survival. The protein may have at least 85, 90, 95, 96, 97, 98, 99 or 100% identity to NCBI Reference No: NP_006130 or a fragment thereof that has stimulatory activity. In some embodiments, a CAR of the invention may comprise one or more sequences corresponding to CD28 from any organism. In some embodiments, a CAR of the invention may comprise a human CD28 sequence or any sub-sequence thereof.

The term “CD3ζ” or alternatively, “Z”, “zeta”, “3zeta”, “3Zeta”, “zeta chain”, “CD3-zeta” or “TCR-zeta” is defined as the protein provided as GenBank Ace. No. BAG36664.1, or the equivalent residues from a non-human species, e.g., mouse, rodent, monkey, ape and the like. A “CD3ζ intracellular signaling domain” or alternatively a “zeta intracellular signaling domain” or a “TCR-zeta intracellular signaling domain” or a “CD3ζ (cytoplasmic domain” is defined as the amino acid residues from the cytoplasmic domain of the CD3ζ (chain, or functional derivatives thereof, that are sufficient to functionally transmit an initial signal necessary for T cell activation. In some embodiments, a CAR of the invention may comprise part or all of a CD3ζ cytoplasmic domain.

As used herein, the term “chimeric antigen receptor” or “CAR” means a protein that when expressed on the surface of a cell allows a CAR expressing cell to recognize its specific protein (antigen), such as on tumor cells, infected cells or cells mediating autoimmune or inflammatory diseases or disorders. Such receptors are also known as chimeric T cell receptors, chimeric immunoreceptors, or artificial T cell receptors. Upon transduction of a cell with a nucleic acid construct encoding a CAR, the cell will recognize the antigen specified by the CAR. A CAR is typically comprised of an ectodomain (extracellular domain) and an endodomain (cytoplasmic domain), separated by a transmembrane domain. The ectodomain, expressed on the surface of the cell, comprises an antigen binding domain or receptor domain, optionally a signal peptide that directs the antigen binding domain into the endoplasmic reticulum for processing, and optionally a spacer (or hinge) region. The antigen binding domain (or receptor domain) comprises peptides that specifically recognize a target antigen. As a non-limiting example, the antigen binding domain can be a single chain antibody, such as an scFv. The spacer region links the antigen binding domain to the transmembrane domain and is designed to be sufficiently flexible to allow the antigen binding domain to orient in a manner that allows antigen recognition. Examples of spacer domains include, but are not limited to, the hinge region from IgG, the CH₂CH₃ region of an immunoglobulin, CD28 hinge, Dap10 hinge, CD8 hinge, and portions of CD3 molecules. The transmembrane domain is a hydrophobic alpha helix, typically, that spans across the lipid bilayer of the cell membrane. An example of a transmembrane domain is the transmembrane domain from CD28, explained in more detail, infra. The endodomain of the CAR is composed of a signal transmitting peptide that transmits an activation signal intracellularly to the cell cytoplasm, thereby stimulating the cell expressing the CAR. The endodomain may include multiple such signaling domains, as explained, infra. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides encoding the CAR are contiguous with each other. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule is the zeta chain associated with the T cell receptor complex. In one aspect, the cytoplasmic signaling domain further comprises one or more functional signaling domains derived from at least one costimulatory molecule as defined below. In one aspect, the CAR comprises a chimeric fusion protein comprising an extracellular antigen binding domain, a transmembrane domain and an intracellular signaling domain comprising a functional signaling domain derived from a stimulatory molecule. In one aspect, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one aspect, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen binding domain (e.g., an scFv) during cellular processing and localization of the CAR to the cellular membrane.

The term “compete”, as used herein with regard to an antibody, means that a first antibody, or an antigen binding fragment (or portion) thereof, binds to an epitope in a manner sufficiently similar to the binding of a second antibody, or an antigen binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the description herein. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

As used herein, the term “co-stimulatory ligand,” includes a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin β receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

The term “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to a protein selected from the group consisting of an MHC class I molecule, TNF receptor proteins, Immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8α, CD8β, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, OX40 (CD134), 4-1BB (CD137), SLAM (SLAMF1, CD150, IPO-3), CD160 (BY55), SELPLG (CD162), DNAM1 (CD226), Ly9 (CD229), SLAMF4 (CD244, 2B4), ICOS (CD278), CEACAM1, CDS, CRTAM, DAP10, GADS, GITR, HVEM (LIGHTR), IA4, ICAM-1, IL2R β, IL2R γ, IL7R α, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and a ligand that specifically binds with CD83.

As used herein, the term “co-stimulatory signal”, refers to a signal, which in combination with a primary signal, such as TCR/CD3 ligation, leads to T cell proliferation and/or upregulation or down regulation of key molecules.

As used herein, the term “cytokine” means a secreted, low-molecular-weight (about 5 to 20 kDa) protein expressed by cells that regulate the nature, intensity, and duration of an immune response by exerting a biological effect on cells that express receptors that bind the cytokine. Cytokines are often pleiotropic, possessing different biological effects when bound by different cell types and can modulate the balance between the humoral and the cell-based (innate) immune response. Cytokines play an important role in activating and stimulating cells of the immune system and other cells. Cytokines are generally divided into four structural families including the hematopoietin family, interferon (IFN) family, chemokine family, and tumor necrosis factor (TNF) family. The term cytokine encompasses interleukins, lymphokines, monokines, interferons, colony stimulating factors, and chemokines. Examples of cytokines include, but are not limited to, IL-1, IL-2, IL-4, IL-7, IL-9, IL-13, IL-15, IL-3, IL-5, granulocyte macrophage colony-stimulating factor (GM-CSF), IL-6, IL-11, IL-12, G-CSF, leukemia inhibitory factor (LIF), IL-10, IL-20, IL-14, INF-α, INF-β, INF-γ, TNF, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β, Epo, Tpo, Flt-3L, Stem Cell Factor (SCF), M-CSF, and MSP. (See, Cameron et al., Madame Curie Bioscience Database [Internet]; Austin (Tex.): Landes Bioscience; 2000-2013). Cytokines may be involved in autocrine signalling, paracrine signalling and/or endocrine signalling as immunomodulating agents. Cytokines are produced by a broad range of cells, including immune cells like macrophages, B lymphocytes, T lymphocytes and mast cells, as well as endothelial cells, fibroblasts, and various stromal cells. “Chemokines” are a family of cytokines generally involved in mediating chemotaxis.

The term “DAP10”, also referred to herein as “D10”, refers to the protein Hematopoietic Cell Signal Transducer, a signal adaptor protein expressed on NK cells and certain T cells that provides co-stimulatory signals that lead to the effector functions of the cells (e.g., cytotoxicity and cytokine secretion). The protein may have at least 85, 90, 95, 96, 97, 98, 99 or 100% identity to GenBank Accession Number: AAH65224.1 or a fragment thereof that has stimulatory activity. In some embodiments, a CAR of the invention may comprise one or more sequences corresponding to DAP10 from any organism. In some embodiments, a CAR of the invention may comprise a human DAP10 sequence or any sub-sequence thereof.

As used herein, the term “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount”, “an amount effective to treat” or a “therapeutically effective amount” refers to the amount of the subject compound that will elicit the biological or medical response of a tissue, system, or subject that is being sought by the researcher, veterinarian, medical doctor or other clinician. In some embodiments, this refers to a dose that is adequate to prevent or treat a disease, condition, or disorder in an individual. In some embodiments, this amount includes that amount of a compound that, when administered, is sufficient to prevent development of, or alleviate to some extent, one or more of the signs or symptoms of the disorder or disease being treated. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, perhaps using the inventive CAR materials in each or various rounds of administration.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system. For example an “endogenous” TCR is one normally or naturally expressed on the surface of a primary T cell.

As used herein, the term “exogenous” refers to any material introduced from or produced outside an organism, cell, tissue or system.

As used herein, the term “expression” is defined as the transcription and/or translation of a particular nucleotide sequence driven by its promoter.

As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “hinge”, “spacer”, or “linker” refers to an amino acid sequence of variable length typically encoded between two or more domains of a polypeptide construct to confer flexibility, improved spatial organization, proximity, etc.

As used herein, the term “homologous” refers to two sequences having similar sequences and analogous function/structure. Sequence “identity” at a position is used to indicate that the position in both of the two compared sequences is occupied by the same base or amino acid monomer subunit, e.g., if a position in each of two DNA molecules is occupied by adenine, then the molecules are identical at that position. The percent of identity between two sequences is a function of the number of matching or homologous positions shared by the two sequences divided by the number of positions compared×100. For example, if 6 of 10 of the positions in two sequences are identical then the two sequences are 60% identical. By way of example, the DNA sequences ATTGCC and TATGGC share 50% sequence identity. Generally, a comparison is made when two sequences are aligned to give maximum sequence identity.

As used herein, “human antibody” means an antibody having an amino acid sequence corresponding to that of an antibody produced by a human and/or which has been made using any of the techniques for making human antibodies known to those skilled in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., Nature Biotechnology, 14:309-314, 1996; Sheets et al., Proc. Natl. Acad. Sci. (USA) 95:6157-6162, 1998; Hoogenboom and Winter, J. Mol. Biol., 227:381, 1991; Marks et al., J. Mol. Biol., 222:581, 1991). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al. Monoclonal Antibodies and Cancer Therapy, Alan R. Liss, p. 77, 1985; Boerner et al., J. Immunol., 147 (1):86-95, 1991; and U.S. Pat. No. 5,750,373.

As used herein, “immune cell” refers to a cell of hematopoietic origin functionally involved in the initiation and/or execution of innate and/or adaptive immune response.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is a primary antibody that is often present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by, causing release of mediators from mast cells and basophils upon exposure to allergen.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

An “intracellular signaling domain,” as the term is used herein, refers to an intracellular portion of a molecule. The intracellular signaling domain generates a signal that promotes an immune effector function of the cell transduced with a nucleic acid sequence comprising a CAR, e.g., a CAR T cell. Examples of immune effector function, e.g., in a CAR T cell, include cytolytic activity and helper activity, including the secretion of cytokines. Intracellular signaling domains include an intracellular signaling domain of a lymphocyte receptor chain, a TCR/CD3 complex protein, an Fc receptor subunit, an IL-2 receptor subunit, CD3ζ, FcR γ, FcR β, CD3 γ, CD3 δ, CD3ε, CD5, CD22, CD79a, CD79b, CD66d, CD278 (ICOS), FcεRI, DAP10, and DAP12.

An “isolated” biological component (such as an isolated chimeric antigen receptor or cell or vector or protein or nucleic acid) refers to a component that has been substantially separated or purified away from its environment or other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis. An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant amount of gene transfer into living cells. Exemplary vectors of the invention are derived from lentiviruses.

The term “linker” as used in the context of an scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises one or more repeats of the amino acid sequence Gly-Gly-Gly-Gly-Ser (SEQ ID NO:2). In one embodiment, the flexible polypeptide linker includes, but is not limited to, (Gly₄Ser)₃.

The term “masked CAR” refers to a CAR expressing cell that further comprises a masking peptide. This masking peptide may prevent off-target cell killing. The masking peptide is often N-terminal to the CAR construct and may block the cell's ability to bind to unintended targets. The masking peptide may be cleaved from the CAR expressing cell when it encounters a tumor thereby allowing the CAR expressing cell to attack its target without killing off-target cells. The nucleic acid constructs of the invention may optionally encode masked CARs.

A “mutation” as defined herein may comprise any substitution, deletion or addition to a polypeptide sequence or nucleic acid sequence. A mutation may be defined with respect to any wildtype or otherwise original sequence from which the mutated sequence is derived or to which the mutated sequence is related. Mutations may be silent mutations, in the case of nucleic acid sequences, or they may result in a change in the encoded polypeptide sequence. Mutations may or may not be conservative. Mutations may or may not produce functional changes in the activity, expression, or other features of the CAR of the invention.

The term “nucleic acid” and “polynucleotide” refer to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism. The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences. In the context of the present invention, the following abbreviations for the commonly occurring nucleic acid bases are used, “A” refers to adenosine, “C” refers to cytosine, “G” refers to guanosine, “T” refers to thymidine, and “U” refers to uridine.

As used herein, the term “nucleic acid construct” refers to a nucleic acid molecule or polynucleotide, which includes a nucleic acid encoding a chimeric antigen receptor and at least one nucleic acid encoding a transcription factor. The transcription factor can be one that mediates cell differentiation resulting in proinflammatory cytokine expression. In some embodiments, the nucleic acid construct is a linear naked molecule or a vector, e.g., a plasmid, a bacmid, a phagemid, a cosmid, a phage, a virus or an artificial chromosome.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or RNA may also include introns to the extent that the nucleotide sequence encoding the protein may in some version contain an intron(s).

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, intrasynovial injection or infusions; and kidney dialytic infusion techniques. In a preferred embodiment, parenteral administration of the compositions of the present invention comprises subcutaneous or intraperitoneal administration.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types, “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “pharmaceutically acceptable carrier” or “excipient” refers to compounds or materials conventionally used in immunogenic compositions during formulation and/or to permit storage.

As used herein the phrase “primary immune cells” or “primary T cells” refers to immune cells, e.g., T cells derived from donors, e.g., human donors which are allogeneic or autologous relative to a recipient which may be modified, e.g., in order to express a CAR, to delete or disrupt TCR expression or function, and the like, and which cells are useful in human therapy. These cells may be passaged during culturing and modification. Such primary immune cells and modified forms thereof may be distinguished from cell lines, e.g., immortalized T cell lines which are unsuitable for use in human therapy.

By “proinflammatory cytokine” as used herein, is meant any one or more cytokines that function in cell signaling and promote system inflammation, which are produced mainly by macrophages and other innate cell responses involved in upregulation of the inflammatory response. A proinflammatory cytokine encompasses cytokines that activate T helper cells (T_{H}1 and T_{H}2 cells). Proinflammatory cytokines include, but are not limited to, for example, IL-1, TNF-α, TL1A (tumor necrosis factor-like ligand), IL-12, INF-γ, IL-6, MCP-1, and CD40-L.

The term “promoter”, as used herein, is defined as a DNA sequence recognized by the synthetic machinery of the cell, or introduced synthetic machinery, required to initiate the specific transcription of a polynucleotide sequence. A “constitutive” promoter is a nucleotide sequence which, when operably linked with a polynucleotide which encodes or specifies a gene product, causes the gene product to be produced in a cell under most or all physiological conditions of the cell. An “inducible” promoter is a nucleotide sequence which, when operably linked with a polynucleotide which encodes or specifies a gene product, causes the gene product to be produced in a cell substantially only when an inducer which activates or “turns on” the promoter is present in the cell. A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “recombinant” means a polynucleotide with semisynthetic or synthetic origin which either does not occur in nature or is linked to another polynucleotide in an arrangement not found in nature.

The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}. The linker may comprise portions of the framework sequences.

A “signal peptide” (also referred to as a signal sequence, targeting signal, localization signal, localization sequence, transit peptide, leader sequence or leader peptide) is a short peptide present at the N-terminus of the majority of newly synthesized proteins that are destined towards the secretory pathway. The core of the signal peptide may contain a long stretch of hydrophobic amino acids. The signal peptide may or may not be cleaved from the mature polypeptide.

The term “signaling domain” refers to the functional portion of a protein which acts by transmitting information within the cell to regulate cellular activity via defined signaling pathways by generating second messengers or functioning as effectors by responding to such messengers.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory ligand,” as used herein, means a ligand that when present on an antigen presenting cell (e.g., an APC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as an immunoreceptor tyrosine-based activation motif or ITAM. Examples of an ITAM containing cytoplasmic signaling sequence that are of particular use in the invention include, but are not limited to, those derived from CD3, common FcRγ (FCER1G), FcγRIIa, FcR β (Fc E R1b), CD3γ, CD3δ, CD3ε, CD79a, CD79b, DAP10, and DAP12.

As used herein, a “substantially purified” cell is a cell that is substantially not associated with, or which is removed from one or more other moieties with which it is normally associated, e.g., it may be free or essentially free of other cell types. By substantially free is intended that the other moieties, e.g., other cells, may still be present, albeit in lesser amounts or percentages absent purification. A substantially purified cell also refers to a cell which has been separated or substantially separated from other cell types with which it is normally associated in its naturally occurring state, i.e., the isolated cell or cells are present in relatively greater numbers or percentages in the composition relative to the cells which are removed as a consequence of the purification. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “subject” is intended to include living organisms in which an immune response can be elicited (e.g., mammals). Examples of subjects include humans, dogs, cats, mice, rats, and transgenic species thereof. The terms “patient,” “subject,” “individual,” and the like are used interchangeably herein, and refer to any animal or cells thereof whether in vitro or in situ, amenable to the methods described herein. In certain non-limiting embodiments, the patient, subject or individual is a human.

By the term “synthetic antibody” as used herein, is meant an antibody which is generated using recombinant DNA technology, such as, for example, an antibody expressed by a yeast. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “T cell” as used herein encompasses any known T cell. For example, T cells are lymphocytes that express a T cell receptor (TCR). T cells mature in the thymus from thymocytes. The term T cell encompasses, for example, helper T cells (T_{H} cells) such as T_{H}1, T_{H}2, T_{H}3, T_{H}17, T_{H}9, and T_{FH} cells; cytotoxic T lymphocyte cells (T_{C} or CTL); memory T cells, such as central memory T cells, effector memory T cells, and resident memory T cells (T_{CM} cells, T_{EM} cells, T_{EMRA} cells, and T_{RM} cells, respectively); suppressor T cells, a type of regulatory T cell (T_{reg} cell); natural killer cells (NKT cells); mucosal associated invariant T cells (MAITs); and γ-δ T cells, for example. T cells can be obtained from a subject, making them a primary T cell. T cells can be immature, allogeneic, autologous, xenogeneic, mortal or immortal.

The “T2A ribosome skip sequence” refers to an amino acid sequence that, when translated, causes cleavage of a nascent polyprotein on the ribosome, allowing for co-expression of multiple genes.

The term “therapeutic” as used herein means a treatment and/or prophylaxis. A therapeutic effect is obtained by suppression, remission, or eradication of a disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny. The exogenous nucleic acid may be introduced stably or transiently into the host cell. Transfection can be achieved any number of known methods, such as retroviral infection and the like.

By the term “transmembrane domain”, what is implied is any three-dimensional protein structure which is thermodynamically stable in a membrane. This may be a single alpha helix, a transmembrane beta barrel, a beta-helix of gramicidin A, or any other structure. Transmembrane helices are usually about 20 amino acids in length. Typically, the transmembrane domain denotes a single transmembrane alpha helix of a transmembrane protein, also known as an integral protein.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”—refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count. Additionally, the terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. The four major types of vectors include plasmids, viral vectors, cosmids, and artificial chromosomes. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “xenogeneic” refers to a graft derived from an animal of a different species.

### EXAMPLES

### Example 1: CAR Constructs

Exemplary anti-B7H6 CAR constructs were created according to the schematics depicted in FIG. 1 and FIG. 2, with mutations as set forth in FIG. 1-3.

Each of the mutant CAR constructs was named as set forth in the fourth column of the table in FIG. 4A and had the following mutations:

JC74 was the “WT” CAR construct, comprising CD28 hinge, transmembrane and cytoplasmic domains;

JC80 was the construct featuring only the expression control gene (mCD19), without a CAR construct;

JC107 has a D190E mutation in motif 1;

JC108 has a Y191A mutation in motif 1;

JC109 has a P196A mutation in motif 2;

JC110 has an R197A mutation in motif 2;

JC135 has a Y209F mutation in motif 3;

JC136 has a PY208AA mutation in motif 3;

JC111 has a KL221 deletion (removal of an extra HindIII site);

JC112 has a C141S mutation in the dimerization motif;

JC114 has the human Dap10 hinge, transmembrane and cytoplasmic domain in place of the CD28 hinge, transmembrane and cytoplasmic domain of the “WT” construct;

JC116 has the human Dap10 hinge, transmembrane and cytoplasmic domain in place of the CD28 hinge, transmembrane and cytoplasmic domain of the “WT” construct and had an additional D57A mutation in the NKG2D binding motif, so it does not associate with NKG2D.

### Example 2: CAR T Cell Expression Testing and Cytokine Production

T cells comprising the various CAR constructs were tested for expression, cytotoxicity, and cytokine production according to the following six assays, numbered as in FIG. 4A and FIG. 4B:

1—% of CAR+ (αmusIgG+) T cells, shown relative to that for JC111 which are made =100 for the top group, and relative to that for JC114 (=100) for the bottom group;

2—cytotoxicity values using % lysis at an E:T ratio of 1:1, calculated using the formula (1-[luminescence experimental sample/luminescence of JC80 (mCD10-only) control×100; shown relative to that for JC111 (=100) for the top group, and relative to that for JC114 (=100) for the bottom group;

3—IFNγ production in T cell:tumor cell cultures (5K T cells+5 K tumor cells); shown relative to that for JC111 (=100) for the top group, and relative to that for JC114 (=100) for the bottom group;

4—IFNγ production following culture of T cells with B7H6-Fc plated at 4 ng/well, shown relative to that for JC111 (=100) for the top group, and relative to that for JC114 (=100) for the bottom group;

5—GMCSF production following culture of T cells with B7H6-Fc plated at 20 ng/well, shown relative to that for JC111 (=100) for the top group, and relative to that for JC114 (=100) for the bottom group; and

6—IL-2 or TNFα amounts following culture of T cells with B7H6-Fc plated at 100 ng/well, shown relative to that for JC111 (=100) for the top group, and relative to that for JC114 (=100) for the bottom group.

Results are provided in FIG. 4A.

### Example 3: CAR T Cell Cytokine Production

Relative cytokine production of CAR T cells was measured as a function of relative CAR expression compared to a CD28-3zeta construct (JC111) for each cytokine. Results are provided in FIG. 5. For each construct, the cytokine productions were normalized to the values for T cells comprising construct JC111 and for the CAR expression on T cells for each construct.

### Example 4: JC135 and JC136 Cytotoxicity

The cytotoxicity of T cells comprising the JC135 and JC136 constructs was measured against OvCAR5 tumor cells (ligand-positive); K562 tumor cells (ligand-positive); and RAM tumor cells (ligand-negative), as illustrated in FIG. 6A-6C. The EC₅₀ values for these cytotoxicity assays are provided in FIGS. 7A-7B.

### Example 5: JC135 and JC136 Cytokine Production

The cytokine production of T cells comprising the JC135 and JC136 constructs was measured in the presence of target cells for the following cytokines: IFNγ, IL-2, GM-CSF, and TNFα. JC111 and JC80 were used as controls. Results are displayed in FIGS. 8A-8F.

### Example 6: JC114 and JC116 Cytotoxicity

The cytotoxicity of T cells comprising the JC114 and JC116 constructs was measured against OvCAR5 tumor cells (ligand-positive); K562 tumor cells (ligand-positive); and RAM tumor cells (ligand-negative), as illustrated in FIG. 9A-9C. JC74 and JC80 were used as controls. The EC₅₀ values for these cytotoxicity assays are provided in FIGS. 10A-10B.

### Example 7: JC114 and JC116 Cytokine Production

The cytokine production of T cells comprising the JC114 and JC116 constructs was measured in the presence of target cells for the following cytokines: IFNγ, IL-2, GM-CSF, and TNFα. JC74 was used as a control. Results are displayed in FIGS. 11A-11D.

### Example 8: CAR T Cells Inhibit Pancreatic Cancer In Vivo

MICA-specific CAR T cells were created by transduction with the JC143 construct: anti-MICA CAR B2-D10h/D10D57ATM/D10cyto-3Zeta. Control T cells were transduced with the JC80 construct (mCD19 only vector). PANC-1 bearing NSG mice were given MICA-specific CAR T cells or control transduced T cells on day 12 and day 26. Tumor burden was assessed by luminescence using a Xenogen imaging device. The PANC-1 tumor cells were engineered to express luciferase. Results are provided in FIG. 12.

What is claimed is:

1. A chimeric antigen receptor (CAR) polypeptide comprising an antigen binding domain, a transmembrane domain, and a cytoplasmic domain, wherein the cytoplasmic domain comprises at least one costimulatory domain, wherein the polypeptide sequence comprises one or more mutations, and wherein the mutation optionally affects one or more functional features of the CAR, selected from the following group:
a. CAR T cell cytokine production;
b, CAR T cell cytotoxicity;
c. target cell specific lysis;
d. CAR dimerization;
e. CAR cytoplasmic domain binding to downstream signaling partners;
f. specificity of CAR T cell cytotoxicity; and
g. CAR surface expression.

2. The CAR polypeptide according to claim 1, wherein the polypeptide comprises one or more sequences derived from CD28, wherein said one or more sequences optionally comprise any one or more of the followings:
(i) one or more of a CD28 hinge domain, a CD28 transmembrane domain, and a CD28 cytoplasmic domain, and/or
(ii) one or more of the following motifs, wherein at least one of said motifs optionally comprises a mutation or deletion:
a. motif 1;
b. motif 2;
c. motif 3;
d. motif 4;
e. dimerization motif;
f. PI3K binding motif;
g. Grb2 binding motif;
h. Gads binding motif;
i. Itk binding motif;
j. LCK-PKCθ binding motif;
k. FilA binding motif;
l. ubiquitin binding motif; and
m. HindIII encoded motif, and/or
(iii) one or more of the followings
a. C141S;
b. D190E;
c. Y191A;
d. P196A;
e. R197A;
f. PY208AA;
g. PYAPP208AYAAA;
h. Y209F; and
i. KL221 deletion.

3. The CAR polypeptide according to claim 1 or 2, wherein the polypeptide comprises one or more sequences derived from DAP10, wherein said one or more sequences optionally comprise one or more of a DAP10 hinge domain, a DAP10 transmembrane domain, and a DAP10 cytoplasmic domain, and/or wherein said transmembrane domain optionally comprises an NKG2D binding motif optionally comprising a mutation, optionally a D57A mutation.

4. The CAR polypeptide according to any one of the foregoing claims, wherein:
(i) the antigen binding domain specifically recognizes any one of: B7H6, MICA, CD19, CD20, CD22, kappa light chain, CD38, receptor-tyrosine-kinase-like orphan receptor 1 (ROR1), CD30, CD33, epithelial glycoprotein (EGP) 40, tumor-associated glycoprotein 72, prostate-specific membrane antigen, prostate stem cell antigen, ganglioside (GD) 3, high molecular weight melanoma-associated antigen, HLA-A1 MAGEA1, ErbB2, mucin (MUC) 1, MUC16, folate receptor-α, CD44v7/8, carbonic anhydrase 9, G250/CAIX, GD2, CD171, nerve cell adhesion molecule, fetal acetylcholine receptor, ErB3/4, epidermal growth factor receptor VIII, carcinoembryonic antigen, EGP2, mesothelin, natural killer group 2 member D ligands, IL-13 receptor α2, HLA-A2 NY-ESO-1, CD44v6, αvβ6 integrin, 8H9, vascular endothelial growth factor receptors, and 5T4, and/or optionally
(ii) the CAR comprises a human, humanized, or chimeric antigen binding domain, optionally wherein the antigen binding domain comprises a human, humanized, or chimeric scFv.

5. The CAR polypeptide according to any one of the foregoing claims, wherein the CAR optionally comprises any one or more of:
(i) a transmembrane domain derived from a protein selected from the group consisting of CD28, CD3ε, CD4, CD5, CD8, CD9, CD16, CD22, CD33, CD37, CD45, CD64, CD80, CD86, CD134, CD137, CD154, DAP10, TCRα, TCRβ, and CD3ζ,
(ii) at least one of the endodomains of one or more of a lymphocyte receptor chain, a TCR/CD3 complex protein, an Fc receptor subunit, an IL-2 receptor subunit, FcRγ, FcRβ, CD3γ, CD3δ, CD3ε, CD5, CD22, CD79a, CD79b, CD66d, CD2, CD4, CD5, CD8α, CD8β, CD28, CD134, CD137, ICOS, CD122, CD132, CD40, CD154, FcεRI, DAP10, DAP12 or CD3ζ, and
(iii) one or more costimulatory endodomains derived from a protein selected from the group consisting of an MHC class I molecule, TNF receptor proteins, Immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8α, CD8β, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, OX40 (CD134), 4-1BB (CD137), SLAM (SLAMF1, CD150, IPO-3), CD160 (BY55), SELPLG (CD162), DNAM1 (CD226), Ly9 (CD229), SLAMF4 (CD244, 2B4), ICOS (CD278), CEACAM1, CDS, CRTAM, DAP10, GADS, GITR, HVEM (LIGHTR), IA4, ICAM-1, IL2R β, IL2R γ, IL7R α, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and a CD83 ligand or any combination of the foregoing.

6. The CAR polypeptide according to any one of the foregoing claims, wherein the CAR optionally comprises:
(i) a CD3ζ stimulatory endodomain,
(ii) a CD3ζ stimulatory endodomain and a CD28 costimulatory endodomain,
(iii) a CD3ζ stimulatory endodomain and a DAP10 costimulatory endodomain or
(iv) a CD3ζ stimulatory endodomain, a CD28 costimulatory endodomain and a DAP10 costimulatory endodomain.

7. A CD28 polypeptide
(i) comprising one or more of the following motifs, wherein at least one of said motifs comprises a mutation or deletion:
a. motif 1;
b. motif 2;
c. motif 3;
d. motif 4;
e. dimerization motif;
f. PI3K binding motif;
g. Grb2 binding motif;
h. Gads binding motif;
i. Itk binding motif;
j. LCK-PKCθ binding motif;
k. FilA binding motif;
l. ubiquitin binding motif; and
m. HindIII encoded motif, and
(ii) optionally wherein said mutation or deletion comprise one, two, three, four, five, six, seven, eight or all nine or more of the following:
a. C141S;
b. D190E;
c. Y191A;
d. P196A;
e. R197A;
f. PY208AA;
g. PYAPP208AYAAA;
h. Y209F; and
i. KL221 deletion.

8. A DAP10 polypeptide comprising a DAP10 transmembrane domain, wherein said domain comprises an NKG2D binding motif comprising a mutation, optionally a D57A mutation.

9. A nucleic acid sequence encoding the CAR according to any one of claims 1-6, the CD28 polypeptide according to claim 7, or the DAP10 polypeptide according to claim 8.

10. A vector comprising the nucleic acid sequence of claim 9, wherein the vector is optionally a DNA, an RNA, a plasmid, a lentivirus vector, an adenoviral vector, a retrovirus vector, or an in vitro transcribed vector.

11. A recombinant cell expressing the CAR polypeptide according to any one of claims 1-6, optionally comprising the nucleic acid sequence according to claim 9, or comprising the vector according to claim 10.

12. The recombinant cell according to claim 11, wherein
(i) the cell is an immune cell, optionally a primary mammalian immune cell, optionally a primary human immune cell, and further optionally
(ii) the cell is selected from a T lymphocyte, a B lymphocyte, a natural killer cell, an eosinophil, an NK/T cell, a macrophage, a cell of myeloid lineage, a dendritic cell, a neutrophilic granulocyte, a monocyte, a T cell progenitor, a CD4+ T cell, a CD8+ T cell, a naive T (TN) cell, an immature T cell, an effector T (TEFF) cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a helper T cell, a cytotoxic T lymphocyte (CTL), a mucosa-associated invariant T (MAIT) cell, a regulatory T (Treg) cell, a helper T cell, a TH1 cell, a TH2 cell, a TH3 cell, a TH17 cell, a TH9 cell, a TH22 cell, a follicular helper T cell, an α/β T cell, a δ/γ T cell, a Natural Killer (NK) cell, a Natural Killer T (NKT) cell, a cytokine-induced killer (CIK) cell, and a lymphokine-activated killer (LAK) cell, optionally selected from primary cells obtained from a human donor or donors.

13. The recombinant cell according to claim 11 or 12, wherein the cell is further modified in order to have one or more of the following properties:
a. eliminate or reduce the expression or functionality of the T cell's endogenous T cell receptor (TCR);
b. express the dominant negative form of the transforming growth factor β (TGFβ) receptor (DNR);
c. overexpress pro-survival signals, reverse anti-survival signals, overexpress Bcl-xL, over-express BCL-2, inhibit the function of cell death genes (optionally Bak or Bax), overexpress hTERT, and/or eliminate Fas expression;
d. evade immunosuppressive mediators;
e. inactivate the expression or functionality of a human leukocyte antigen (HLA) gene or HLA regulator gene product;
f. comprise a homing mechanism;
g. express a protein that is capable of triggering cell suicide or elimination; and
h. express a protein whose expression allows for selection of cells expressing the CAR polypeptide.

14. The recombinant cell according to any one of claims 11-13, wherein the cell is engineered to express another CAR, wherein said other CAR comprises an antigen binding domain or receptor, a transmembrane domain, and one or more of an immune signaling or costimulatory endodomain.

15. A therapeutic or pharmaceutical composition comprising a therapeutically or diagnostically effective amount of the recombinant cell according to any one of claims 11-14, optionally further comprising a pharmaceutically acceptable carrier, diluent or excipient.

16. A method of immune therapy comprising administering to a subject a therapeutically effective amount of the CAR polypeptide according to any one of claims 1-6, nucleic acid according to claim 9, vector according to claim 10, recombinant cell according to any one of claims 11-14, or composition according to claim 15, wherein optionally the method is used to treat a human disease or condition, further optionally cancer or another proliferative disease or condition.

17. The method according to claim 16, wherein the treatment comprises adoptive cell therapy (ACT) using immune cells harvested from the subject or from one or more donors, wherein the ACT optionally comprises:
(a) isolating primary immune cells from the subject or from one or more donors,
(b) transducing the primary immune cells with the nucleic acid encoding a CAR polypeptide according to any one of claims 1 to 6,
(c) expressing the CAR in the transduced primary immune cells, and
(d) delivering the transduced immune cells into the subject, and
optionally further comprises
(e) stimulating and/or expanding the immune cells prior to delivering the transduced immune cells to the subject.

18. A method for treating cancer comprising delivering to a subject in need of treatment an effective amount of the CAR polypeptide according to any one of claims 1-6, the nucleic acid according to claim 9, the vector according to claim 10, the recombinant cell according to any one of claims 11-14, or the composition according to claim 15, thereby treating the cancer, optionally wherein the treatment of cancer is measured by a decrease in tumor cell burden or by an increase in survival.

19. A composition comprising a therapeutically effective amount of the CAR polypeptide according to any one of claims 1-6, a nucleic acid according to claim 9, a vector according to claim 10, a recombinant cell according to any one of claims 11-14, or a composition according to claim 15 for use in immune therapy in a subject in need thereof, wherein optionally the subject is human and has a disease or condition, further optionally cancer or another proliferative disease or proliferative condition.

20. The composition according to claim 19, wherein the use comprises adoptive cell therapy (ACT) using immune cells harvested from the subject or from one or more donors, wherein the ACT optionally comprises:
(f) isolating primary immune cells from the subject or from one or more donors,
(g) transducing the primary immune cells with the nucleic acid encoding a CAR polypeptide according to any one of claims 1 to 6,
(h) expressing the CAR in the transduced primary immune cells, and
(i) delivering the transduced immune cells into the subject, and
optionally further comprises
(j) stimulating and/or expanding the immune cells prior to delivering the transduced immune cells to the subject.

21. A composition comprising a therapeutically effective amount of the CAR polypeptide according to any one of claims 1-6, a nucleic acid according to claim 9, a vector according to claim 10, a recombinant cell according to any one of claims 11-14, or a composition according to claim 15 for use in treating cancer in a subject in need thereof, optionally wherein the treatment results in a decrease in tumor cell burden and/or an increase in survival.

22. Use of a therapeutically effective amount of the CAR polypeptide according to any one of claims 1-6, a nucleic acid according to claim 9, a vector according to claim 10, a recombinant cell according to any one of claims 11-14, or a composition according to claim 15 in the preparation of a medicament for use in immune therapy in a subject in need thereof, wherein optionally the subject is human and has a disease or condition, further optionally cancer or another proliferative disease or proliferative condition.

23. The use of claim 22, which comprises adoptive cell therapy (ACT) using immune cells harvested from the subject or from one or more donors, wherein the ACT optionally comprises:
(k) isolating primary immune cells from the subject or from one or more donors,
(l) transducing the primary immune cells with the nucleic acid encoding a CAR polypeptide according to any one of claims 1 to 6,
(m) expressing the CAR in the transduced primary immune cells, and
(n) delivering the transduced immune cells into the subject, and
optionally further comprises
(o) stimulating and/or expanding the immune cells prior to delivering the transduced immune cells to the subject.

24. Use of a therapeutically effective amount of the CAR polypeptide according to any one of claims 1-6, a nucleic acid according to claim 9, a vector according to claim 10, a recombinant cell according to any one of claims 11-14, or a composition according to claim 15 in the preparation of a medicament for use in treating cancer in a subject in need thereof, optionally wherein the treatment results in a decrease in tumor cell burden and/or an increase in survival.

25. A kit comprising the CAR polypeptide according to any one of claims 1-6, nucleic acid construct according to claim 9, vector according to claim 10, recombinant cell according to claims 11-14, or composition according to claim 15.

26. A method of manufacturing a chimeric antigen receptor (CAR) immune cell, which comprises:
a. obtaining immune cells, optionally T cells or NK cells, e.g. primary human T cells or NK cells; and
b. transducing the immune cells with a vector comprising a nucleic acid that encodes the CAR polypeptide according to any one of claims 1-6.


SEQUENCE LISTING (8 sequences)

SEQ ID NO: 1 - protein, 19 aa, Artificial Sequence
  tyrosine-based activation motif (ITAM)
  misc_feature (1)..(1): Xaa denotes Asp or Glu
  misc_feature (2)..(3): Xaa can be any naturally occurring amino acid
  misc_feature (5)..(6): Xaa can be any naturally occurring amino acid
  misc_feature (7)..(7): Xaa denotes Ile or Leu
  misc_feature (8)..(15): Xaa denotes 6 to 8 amino acid residues, wherein the amino acid can be any amino acid residue, where one or two positions can be absent
  misc_feature (17)..(18): Xaa can be any naturally occurring amino acid
  misc_feature (19)..(19): Xaa denotes Ile or Leu
XXXYXXXXXXXXXXXYXXX

SEQ ID NO: 2 - protein, 5 aa, Artificial Sequence
  flexible polypeptide linker
GGGGS

SEQ ID NO: 3 - protein, 90 aa, Artificial Sequence
  anti-B7H6 CAR comprising a CD28 co-stimulatory domain
ASVKGKHLCPSPLFPGPSKPFWVLVVVGGVLACYSLLVTVAFIIFWVRSKRSRLLHSDYMNMTPRRPGPTRKHYQPYAPPRDFAAYRSKL

SEQ ID NO: 4 - protein, 77 aa, Artificial Sequence
  anti-B7H6 CAR with a DAP10 co-stimulatory domain
ASQTTPGERSSLPAFYPGTSGSCSGCGSLSLPLLAGLVAADAVASLLIVGAVFLCARPRRSPAQEDGKVYINMPGRG

SEQ ID NO: 5 - protein, 5 aa, Artificial Sequence
  Motif 1
DYMNM

SEQ ID NO: 6 - protein, 4 aa, Artificial Sequence
  Motif 2
PRRP

SEQ ID NO: 7 - protein, 4 aa, Artificial Sequence
  Motif 3
PYAP

SEQ ID NO: 8 - protein, 5 aa, Artificial Sequence
  hDap10 NKG2D binding
VAADA